(19) United States
(12) Patent Application Publication
(10) Pub. No.: US 2023/0270749 A1
(43) Pub. Date: Aug. 31, 2023

(54) THERMO-SENSITIVE PERMEATION ENHANCING FORMULATIONS FOR DRUG DELIVERY

(71) Applicant: Children's Medical Center Corporation, Boston, MA (US)

(72) Inventors: Daniel S. Kohane, Newton, MA (US); Zipei Zhang, Boston, MA (US)

(73) Assignee: Children's Medical Center Corporation, Boston, MA (US)

(21) Appl. No.: 18/019,302

(22) PCT Filed: Aug. 2, 2021
(86) PCT No.: PCT/US2021/044140

§ 371 Date: Feb. 2, 2023

Related U.S. Application Data
(60) Provisional application No. 63/060,520, filed on Aug. 3, 2020.

(51) Int. Cl. A61K 31/546; A61P 31/04; A61P 27/16; A61K 9/06; A61K 47/10; A61K 47/20; A61K 47/22; A61K 47/06; A61K 31/496

(52) U.S. Cl. CPC A61K 31/546; A61P 31/04; A61P 27/16; A61K 9/06; A61K 47/10; A61K 47/20; A61K 47/22; A61K 47/06; A61K 31/496

(57) ABSTRACT

The present disclosure provides compositions and methods for delivery of therapeutic agents across a barrier. The compositions include a therapeutic agent (e.g., antimicrobial agent, antibiotic), a permeation enhancer comprising sodium dodecyl sulfate, limonene, and/or bupivacaine which increases the flux of the therapeutic agent across the barrier, and a matrix forming agent that is a poloxamer (e.g., poloxamer P188), wherein the composition comprises: between about 0.2% and 3.2% wt/vol of a permeation enhancer that is sodium dodecyl sulfate; when present, between about 0.2% and 2.0% wt/vol of a permeation enhancer that is bupivacaine; between about 0.5% and 7.0% wt/vol of a permeation enhancer that is limonene; between approximately 18-62% P188; and the composition forms a gel at temperatures above a phase transition temperature that is less than about 37° C. The matrix forming agent forms a gel at a suitable gelation temperature and rheological properties for use in drug delivery. Methods of treatment and/or delivery, uses, and kits for the compositions described herein are also provided.

### RELATED APPLICATIONS

This application claims the benefit under 35 U.S.C. § 119(e) to U.S. Provisional application, U.S. Ser. No. 63/060,520, filed Aug. 3, 2020, which is incorporated herein by reference.

### GOVERNMENT SUPPORT

This invention was made with government support under 5R01DC015050-04 awarded by the National Institutes of Health. The government has certain rights in the invention.

### BACKGROUND

Acute Otitis Media (AOM) is one of the most common childhood diseases, accounting for over 20 million physician visits each year in the U.S. Recurrence is also common, with one third of children having six or more episodes of AOM by the age of seven. Up to 80% of children with AOM have mild to severe pain during the onset of the infection, of which about 40% have severe pain. The first 24 to 48 hours are considered to be the most painful period of AOM but about 30% of the children have pain for 3-7 days. Consequently, many AOM guidelines recommend the use of analgesics as an essential part of the treatment. AOM commonly causes pain and distress in children.

Present treatment of ear infections consists of systemic oral antibiotics, a treatment which requires multiple doses over 5-10 days and systemic exposure to antibiotics. The rise in antibiotic resistance, coupled with the many multifactorial etiology of OM pose difficulties in diagnosis and treatment of OM. Furthermore, current treatment presents a number of drawbacks including patient compliance issues due to gastrointestinal side effects, lack of an effective concentration of drug at the site of infection, and the potential for opportunistic infections. Even after acute signs of infection subside, generally within 72 hours, the root cause of the infection may persist for the remainder of the treatment, and beyond, even up to 2 months. Thus, making compliance with a physician's prescription important to prevent reoccurrence of infection.

Local, sustained delivery of active therapeutics directly to the middle ear for the treatment of OM could allow for much higher concentrations of the drug in the middle ear than from systemic administration, while minimizing systemic exposure and its adverse effects. However, the tympanic membrane (TM), while only 10 cell-layers thick, presents a barrier that is largely impermeable to all but the smallest, moderately hydrophobic molecules. Despite being the thinnest layer of skin, it is still a barrier to trans-tympanic membrane diffusion. Therefore, the direct treatment of middle ear infections is problematic. The shortcomings of the current treatment of ear diseases, such as middle ear infections, suggest the need for a new composition delivery system with treatment which is noninvasive and direct acting. Additionally, the use of FDA-approved poloxamers within a thermosensitive composition delivery system forming a hydrogel under suitable conditions sis desirable.

### SUMMARY

In a composition provided herein, the therapeutic agents and permeation enhancers are combined with matrix forming agents, to form compositions which form a hydrogel under suitable conditions. Such conditions may include an increase in temperature during administration (e.g., in the ear canal), or following mixing of two components of the composition or matrix-forming agent. The matrix forming agent is a compound or mixture of compounds that forms a gel after administration. The compositions are generally liquid at ambient conditions, however, once administered to a subject, the matrix forming agent or combination of matrix forming agents causes a phase transition to a hydrogel, at temperatures slightly below room temperature (e.g., between about 20.0° C. and about 30.0° C., for example, at approximately 24° C.). Hydrogels have a highly porous structure that allows for the loading of drugs and other small molecules, and subsequent drug elution out of the gel creates a high local concentration in the surrounded tissues over an extended period. In certain embodiments, the drugs are loaded in the liquid composition. Hydrogels can conform and adhere to the shape of the surface to which they are applied and tend to be biocompatible.

In one aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a block copolymer comprising poloxamer P188;
wherein:
the permeation enhancer or combination of permeation enhancers comprises sodium dodecyl sulfate, limonene, and/or bupivacaine, and
the sodium dodecyl sulfate comprises between about 0.2% and 3.2% of the composition by weight per volume composition;
when present, the bupivacaine comprises between about 0.2% and 2.0% of the composition by weight per volume composition;
the limonene comprises between about 0.5% and 7.0% of the composition by weight per volume composition; and
the poloxamer P188 comprises between about 18% and about 62% of the composition by weight per volume composition;
wherein: the composition forms a gel at temperatures above a phase transition temperature; and
the phase transition temperature is less than about 37° C.
   In certain embodiments, provided herein are compositions wherein:
(c) the block copolymer is poloxamer P188;
the permeation enhancer or combination of permeation enhancers comprises sodium dodecyl sulfate, limonene, and/or bupivacaine, and
the sodium dodecyl sulfate comprises between about 0.5% and 3.2% of the composition by weight per volume composition;
the bupivacaine comprises between about 0.25% and 0.75% of the composition by weight per volume composition;
the limonene comprises between about 1.5% and 6.0% of the composition by weight per volume composition; and
the poloxamer P188 comprises between about 20% and about 50% of the composition by weight per volume composition;
wherein: the composition forms a gel at temperatures above a phase transition temperature; and
the phase transition temperature is between about 20° C. and about 37° C.
   In certain embodiments, provided herein are compositions comprising:
about 1.0% wt/vol of sodium dodecyl sulfate;
about 0.5% wt/vol of bupivacaine;
about 2.0% wt/vol of limonene; and
about 45.0% wt/vol of poloxamer P188.
   In certain embodiments, provided herein are compositions wherein
about 1.0% wt/vol of sodium dodecyl sulfate;
about 0.5% wt/vol of bupivacaine;
about 2.0% wt/vol of limonene;
about 45.0% wt/vol of poloxamer P188; and
about 2.0-5.0% wt/vol of ciprofloxacin.
   In certain embodiments, the composition comprises the following combination:
about 1.0% wt/vol of sodium dodecyl sulfate;
about 0.5% wt/vol of bupivacaine;
about 2.0% wt/vol of limonene; and
about 45.0% wt/vol of poloxamer P188; and
about 2.0-5.0% wt/vol of therapeutic agent (e.g., antibiotic).
   In certain embodiments, the composition comprises the following combination:
about 1.0% wt/vol of sodium dodecyl sulfate;
about 2.0% wt/vol of limonene; and
about 45.0% wt/vol of poloxamer P188; and
about 2.0-5.0% wt/vol of therapeutic agent (e.g., antibiotic).

In another aspect, provided herein are methods and/or uses for treating a disease (e.g., an infectious disease, ear disease, bacterial infection) and/or a condition associated with the disease (e.g., pain associated with an infectious disease, ear disease, bacterial infection) comprising administering a composition comprising a therapeutic agent (e.g., antibiotic), permeation enhancers, and a matrix forming agent, as described herein, to a subject in need thereof.

In certain embodiments, the composition is administered into the ear canal or to the tympanic membrane. In certain embodiments, the disease is otitis media. In certain embodiments, the disease is an ear infection. In certain embodiments, the disease is a bacterial infection (e.g., a H. influenzae, S. pneumoniae, or M. catarhallis infection). In another aspect, provided herein are uses of compositions described herein to treat and/or prevent a disease or condition (e.g., an infectious disease, ear disease, bacterial infection) in a subject in need thereof, the use comprising administering to the subject a therapeutically effective amount of compositions described herein.

In another aspect, provided herein are pharmaceutical compositions comprising a composition described herein, and optionally a pharmaceutically acceptable excipient. In certain embodiments, the pharmaceutical compositions comprise a therapeutically effective amount of the composition for use in treating a disease in a subject in need thereof. In an additional aspect, provided herein are methods for delivering a composition described herein, the method comprising administering into an ear canal of a subject the composition, wherein the composition contacts the surface of a tympanic membrane. The composition may be administered with an eye dropper, syringe, or catheter (e.g., angiocatheter).

In an additional aspect, provided herein are kits comprising a container, a composition described herein, and instructions for administering the composition to a subject in need thereof. The kit may further comprise a device for administration of the composition to a subject, such as a dropper, syringe, catheter, or combination thereof.

The compositions, composition components (e.g., matrix forming agents, therapeutic agents, and permeation enhancers), methods, kits, and uses of the present disclosure may also incorporate any feature described in: Khoo et al., Biomaterials, (2013) 34, 1281-8; U.S. Pat. No. 8,822,410; U.S. patent application Ser. No. 12/993,358, filed May 19, 2009; U.S. patent application Ser. No. 11/734,537; filed Apr. 12, 2007; WIPO Patent Application No. PCT/US2009/003084, filed May 19, 2009, and WIPO Patent Application No. PCT/US2007/009121, filed Apr. 12, 2007, each of which is incorporated herein by reference.

The details of certain embodiments of the disclosure are set forth in the Detailed Description of Certain Embodiments, as described below. Other features, objects, and advantages of the disclosure will be apparent from the Definitions, Examples, Figures, and Claims.

### Definitions

### Chemistry Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). In some embodiments, an alkyl group has 2 to 10 carbon atoms (“C₂₋₁₀ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “polysaccharide” refers to a polymer composed of long chains of carbohydrate or monosaccharide units, or derivatives thereof (e.g., monosaccharides modified to comprise cross-linkable functional groups). Exemplary polysaccharides include, but are not limited to, glycans, glucans, starches, glycogens, arabinoxylans, celluloses, hemicelluloses, chitins, pectins, dextrans, pullulans, chrysolaminarins, curdlans, laminarins, lentinans, lichenins, pleurans, zymosans, glycosaminoglycans, dextrans, hyaluronic acids, chitosans, and chondroitins. The monosaccharide monomers of polysaccharides are typically connected by glysolidic linkages. Polysaccharides may be hydrolyzed to form oligosaccharides, disaccharides, and/or mono saccharides. The term “carbohydrate” or “saccharide” refers to an aldehydic or ketonic derivative of polyhydric alcohols. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. The term carbohydrate also includes other natural or synthetic stereoisomers of the carbohydrates described herein.

These and other exemplary substituents are described in more detail in the Detailed Description, Examples, and Claims. The disclosure is not intended to be limited in any manner by the above exemplary listing of substituents.

### Other Definitions

Animal: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.
Approximately or About: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).
Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.
Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Optically transparent: As used herein, the term “optically transparent” refers to substances through which light passes through with little or no light being absorbed or reflected. In some embodiments, optically transparent refers to substances through which light passes through with no light being absorbed or reflected. In some embodiments, optically transparent refers to substances through which light passes through with little light being absorbed or reflected. In some embodiments, an optically transparent substance is substantially clear. In some embodiments, an optically transparent substance is clear.
Effective amount: In general, the “effective amount” of an active agent refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the patient. The effective amount of a compound used to treat infection is the amount needed to kill or prevent the growth of the organism(s) responsible for the infection.
In vitro: As used herein, the term “in vitro” refers to events that occur in an artificial environment, e.g., in a test tube or reaction vessel, in cell culture, etc., rather than within an organism (e.g. animal, plant, and/or microbe).
In vivo: As used herein, the term “in vivo” refers to events that occur within an organism (e.g. animal, plant, and/or microbe).
Suffering from: An individual who is “suffering from” a disease, disorder, and/or condition has been diagnosed with or displays one or more symptoms of the disease, disorder, and/or condition.
Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.
Therapeutic agent: Also referred to as a “drug” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18th Ed. (2006) following its publication, Mark H. Beers and Robert Berkow (Eds.), Merck Publishing Group, or, in the case of animals, The Merck Veterinary Manual, 9^{th} ed., Kahn, C. A. (Ed.), Merck Publishing Group, 2005.
Diagnostic agent: As used herein, the term “diagnostic agent” refers to an agent that is administered to a subject to aid in the diagnosis of a disease, disorder, or condition. In some embodiments, a diagnostic agent is used to define and/or characterize the localization of a pathological process. Diagnostic agents include X-ray contrast agents, radioactive isotopes, and dyes.
Surfactant: As used herein, the term “surfactant” refers to any agent which preferentially absorbs to an interface between two immiscible phases, such as the interface between water and an organic solvent, a water/air interface, or an organic solvent/air interface. Surfactants usually possess a hydrophilic moiety and a hydrophobic moiety. Surfactants may also promote flux of a therapeutic or diagnostic agent across a biological membrane, e.g., a tympanic membrane.
Terpenes: As used herein, the term “terpene” refers to any agent derived, e.g., biosynthetically, or thought to be derived from unit(s) of isoprene (a five carbon unit). For example, isoprene units of terpenes may be linked together to form linear chains or they may be arranged to form rings. Typically, the terpenes disclosed herein promote flux of a therapeutic or diagnostic agent across a biological membrane, e.g., a tympanic membrane. Terpenes may be naturally derived or synthetically prepared.

The terms “composition” and “formulation” are used interchangeably.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIG. 1 shows a schematic representation of the in-situ gel of an exemplary formulation (containing antibiotics, for example, ciproflaxin; chemical permeation enhancers, for example, limonene, sodium dodecyl sulfate, and bupivacaine; and poloxamer-based thermosensitive hydrogel) at the tympanic membrane (TM). Antibiotics from the hydrogel formulation shows flux across the TM, and into the infected middle ear cavity.

FIG. 2 shows the viscoelastic properties (storage modulus (G′) and loss modulus (G″) (as moduli, in Pa)) of the different depicted compositions of 45%[Poloxamer P188], 3CPEs-45%[Poloxamer P188], and 18%[Poloxamer P407], as a function of increasing temperature. The term “3CPEs” refers to the following group of three chemical permeation enhancers (“CPE's”): 2% limonene (“LIM”), 1% sodium dodecyl sulfate (“SDS”), and 0.5% bupivacaine (“BUP”). Data are means±SD (n=4). Poloxamer P188 is referred to throughout as “P188,” and Poloxamer P407 is referred to as “P407.”

FIGS. 3A-3C show the effects of P188 concentration on the rheological properties of formulation (3CPEs-[P188]). FIG. 3A shows the effect of poloxamer concentration on gelling temperature and gelation time, FIG. 3B shows the effect of poloxamer concentration on viscosity at room temperature and storage modulus at 37° C., and FIG. 3C shows shear rate dependent shear stress of formulation with different poloxamer concentration. 3CPEs: 2% LIM, 1% SDS and 0.5% BUP. Data are means±SD (n=4).

FIG. 4 shows liquid-to-gel transition of 3CPEs-[P188] formulation from room temperature (20° C.) to body temperature (37° C.).

FIGS. 5A-5C show the effect of limonene concentration on the rheological properties of formulation (Lim-45% [P188]). FIG. 5A shows the effect of limonene concentration on gelling temperature and gelation time, FIG. 5B shows the effect of limonene concentration on viscosity at room temperature and storage modulus at 37° C., and FIG. 5C shows the shear rate dependent shear stress of formulation as a function of limonene concentration. Data are means±SD (n=4).

FIGS. 6A-6C show the effect of SDS (FIG. 6A) and bupivacaine (FIG. 6B, FIG. 6C) concentration on viscoelastic properties of 45% [P188], as a function of temperature.

FIGS. 7A-7C show the effect of SDS concentration on the rheological properties of formulation (2% LIM-[P188]). FIG. 7A shows the effect of SDS concentration on gelling temperature and gelation time, FIG. 7B shows the effect of SDS concentration on viscosity at room temperature and storage modulus at 37° C., and FIG. 7C shows the shear rate dependent shear stress of formulations with different SDS concentration. Data are means±SD (n=4).

FIG. 8 shows the cumulative ex vivo transfer of ciprofloxacin (Cip) across the TM into a receiving chamber. 3CPEs: 2% LIM, 1% SDS and 0.5% BUP. Data are means±SD (n=4).

FIG. 9 shows the concentration of ciprofloxacin over time in the middle ear fluid of the animals with OM from NTHi treated with different formulation. n=3 for the formulation containing 4% Cip-3CPEs; n=4 for the formulation containing 12% (w/v) poloxamer 407-polybutylphosphoester (4% Cip-3CPE-[P407-PBP]); and n=6 for the formulation containing 45% P188-3CPE. 3CPEs: 2% LIM, 1% SDS and 0.5% BUP. Data are means±SD.

FIGS. 10A-10B. FIG. 10A shows the percentage of animals with OM (defined as nonzero CFU values in their middle ear fluid aspirates) after administration of different formulations, and FIG. 10B shows the time course of bacterial CFU from middle ear fluid from animals with OM from NTHi treated with different formulation. n=3 for the formulation containing 4% Cip and 4% Cip-3CPE; n=4 for the formulation containing 12% (w/v) poloxamer 407-polybutylphosphoester (4% Cip-3CPE-[P407-PBP]); and n=6 for the formulation containing 45% P188-3CPEs. 3CPEs: 2% LIM, 1% SDS and 0.5% BUP. Data are means±SD.

FIG. 11 shows the in vivo effect on tissue for different formulation. The H&E-stained sections were obtained from healthy TMs and of TMs after 7 days of otitis media (OM), without or after treatment with the formulation containing 4% Cip and 45% P188-3CPEs.

FIGS. 12A-12C show the effect of limonene concentration on the rheological properties of formulation (45% [P188]-Lim). FIG. 12A shows the effect of limonene concentration on gelling temperature and gelation time, FIG. 12 B shows the effect of limonene concentration on viscosity at room temperature and storage modulus at 37° C., and

FIG. 12 C shows the shear rate dependent shear stress of formulation as a function of limonene concentration. Data are means±SD (n=4).

FIGS. 13A-13B show the effect of SDS (FIG. 13A) and bupivacaine (FIG. 13B) concentration on viscoelastic properties of 45% [P188], as a function of temperature.

FIGS. 14A-14C show the effect of SDS concentration on the rheological properties of formulation (45% [P188]-2% Lim-SDS). FIG. 14A shows the effect of SDS concentration on gelling temperature and gelation time, FIG. 14B shows the effect of SDS concentration on viscosity at room temperature and storage modulus at 37° C., and FIG. 14C shows the shear rate dependent shear stress of formulations with different SDS concentration. Data are means±SD (n=4).

FIG. 15 shows the effect of bupivacaine concentration on the rheological properties of formulation (45% [P188]-2% Lim-Bup).

FIGS. 16A-16C. FIG. 16A shows the cumulative Ex vivo transfer of ciprofloxacin (Cip) across the tympanic membrane into a receiving chamber (n=4). FIG. 16B shows a time course of bacterial CFU from middle ear fluid from animals with OM from NTHi treated with different formulation. FIG. 16C shows the percentage of animals with OM (defined as nonzero CFU values in their middle ear fluid aspirates) after administration of different formulations. 3CPEs: 2% LIM, 1% SDS and 0.5% BUP. Data are means±SD.

FIGS. 17A-17C show the concentration of different CPEs including (FIG. 17A) LIM, (FIG. 17B) SDS and (FIG. 17C) BUP over time in the middle ear fluid of the animals with otitis media (OM) from NTHi treated with 45% [P188]-3CPEs. 3CPEs: 2% LIM, 1% SDS and 0.5% BUP. Data are means±SD.

FIG. 18 shows in vivo effect on tissue for different formulation. The H&E-stained sections were obtained from healthy TMs and of TMs after 7 days of otitis media (OM), without or after treatment with the formulation containing 4% Cip and 45% [P188]-3CPEs.

FIG. 19 shows the optical images of tympanic membrane excised from healthy chinchillas after administration of 18% [P407] or 45% [P188]-3CPEs formulation for 21 days. 3CPEs: 2% LIM, 1% SDS and 0.5% BUP.

FIG. 20 shows the storage modulus (G′) of different thermoresponsive gels (P407; P188-3CPEs) at 37° C. 3CPEs: 2% LIM, 1% SDS and 0.5% BUP.

FIG. 21 shows the rheology (storage modulus G′; loss modulus G″) of formulations (poloxamers and different concentrations of CPEs) as a function of temperature.

FIGS. 22A-22D show the rheology rheology (storage modulus G′; loss modulus G″) of formulations (poloxamer 331 and specified concentrations of CPEs) as a function of temperature.

FIGS. 23A-23B show the effect of SDS concentration on gelling temperature, gelation time (FIG. 23A), viscosity, storage modulus (FIG. 23B) at 37° C. and shear stress of the 45% [P188]-2% LIM at fixed shear rate of 100 s-1. 3CPEs: 2% LIM, 1% SDS and 0.5% BUP. Data are means±SD (n=4).

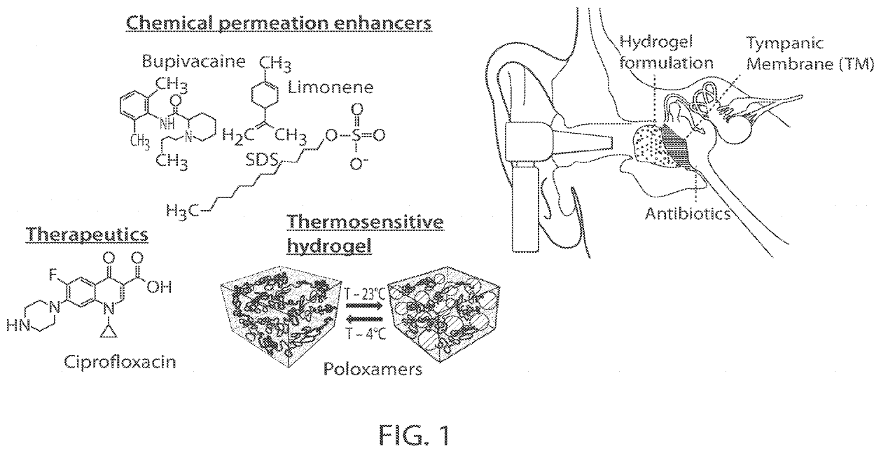

### DETAILED DESCRIPTION OF CERTAIN EMBODIMENTS OF THE DISCLOSURE

Provided herein are compositions and methods for administering a therapeutic agent to a subject through a barrier. In some embodiments, the composition is for administering a therapeutic agent to the ear of a subject, and the barrier is a tympanic membrane. The compositions and methods provide for the efficient delivery of the agent to the middle and/or inner ear of the subject. In one aspect, the composition comprises a combination of a permeation enhancer, a therapeutic agent, and a matrix forming agent. In various aspects, the composition is a single application composition for localized, sustained delivery of a therapeutic agent or a combination of therapeutic agents across the tympanic membrane. In various aspects, the composition is a multiple application composition for localized, sustained delivery of a therapeutic agent across the tympanic membrane. The compositions and methods described herein are particularly useful in treating otitis media and/or pain associated with otitis media by providing sustained release and delivery of an antibiotic to the middle ear. In certain embodiments, the compositions disclosed herein are liquid at lower temperatures but form a gel at temperatures below body temperature.

In one aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; wherein:
the permeation enhancer or combination of permeation enhancers comprises sodium dodecyl sulfate, limonene, and/or bupivacaine, and
the sodium dodecyl sulfate comprises between about 0.2% and 3.2% of the composition by weight per volume composition;
the limonene comprises between about 0.5% and 7.0% of the composition by weight per volume composition; and
the poloxamer P188 comprises between about 18% and about 62% of the composition by weight per volume composition;
wherein:
the composition forms a gel at temperatures above a phase transition temperature; and
the phase transition temperature is less than about 37° C.

In one aspect, provided herein are compositions comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a block copolymer comprising poloxamer P188;
wherein:
the permeation enhancer or combination of permeation enhancers comprises sodium dodecyl sulfate, limonene, and/or bupivacaine, and
the sodium dodecyl sulfate comprises between about 0.2% and 3.2% of the composition by weight per volume composition;
when present, the bupivacaine comprises between about 0.2% and 2.0% of the composition by weight per volume composition;
the limonene comprises between about 0.5% and 7.0% of the composition by weight per volume composition; and
the poloxamer P188 comprises between about 18% and about 62% of the composition by weight per volume composition;
wherein:
the composition forms a gel at temperatures above a phase transition temperature; and
the phase transition temperature is less than about 37° C.
   In certain embodiments, all of the following parameters are met:
(c) the block copolymer is poloxamer P188;
the permeation enhancer or combination of permeation enhancers comprises sodium dodecyl sulfate, limonene, and/or bupivacaine, and
the sodium dodecyl sulfate comprises between about 0.5% and 3.2% of the composition by weight per volume composition;
the bupivacaine comprises between about 0.25% and 0.75% of the composition by weight per volume composition;
the limonene comprises between about 1.5% and 6.0% of the composition by weight per volume composition; and
the poloxamer P188 comprises between about 20% and about 50% of the composition by weight per volume composition;
wherein:
the composition forms a gel at temperatures above a phase transition temperature; and
the phase transition temperature is between about 20° C. and about 37° C.
   In certain embodiments, all of the following parameters are met:
(c) the block copolymer is poloxamer P188;
the permeation enhancer or combination of permeation enhancers comprises sodium dodecyl sulfate and limonene, and
the sodium dodecyl sulfate comprises between about 0.5% and 3.2% of the composition by weight per volume composition;
the limonene comprises between about 1.5% and 6.0% of the composition by weight per volume composition; and
the poloxamer P188 comprises between about 20% and about 50% of the composition by weight per volume composition;
wherein:
the composition forms a gel at temperatures above a phase transition temperature; and
the phase transition temperature is between about 20° C. and about 37° C.

### Permeation Enhancers

A permeation enhancer refers to any agent that optionally increases the flux of a therapeutic agent across a barrier (e.g., membrane, layer of cells). In some embodiments, the barrier is skin. In some embodiments, the permeation enhancer refers to any agent that increases the flux of a therapeutic agent across a barrier (e.g., membrane, layer of cells). In some embodiments, the barrier is the tympanic membrane. In some embodiments, the barrier is the tympanic membrane and not the nerve. In some embodiments, the barrier is not the nerve. The permeation enhancer facilitates delivery of the therapeutic agent (e.g., agents that have a therapeutic benefit in the ear) into the middle and/or inner ear. In certain embodiments, the composition comprises a permeation enhancer or combination of permeation enhancers that is present in an amount effective to increase the flux of the therapeutic agent across a barrier compared to the reference composition (e.g., the composition without the permeation enhancer). In certain embodiments, the permeation enhancer or combination of permeation enhancers is present in an amount effective to increase the flux of the therapeutic agent across a barrier compared to the reference composition (e.g., the composition without the permeation enhancer) by at least about 1.05 fold, at least about 1.10 fold, at least about 1.2 fold, at least about, at least about 1.3 fold, at least about 1.4 fold, at least about 1.5 fold, at least about 1.6 fold, at least about 1.7 fold, at least about 1.8 fold, or at least about 1.9 fold. In certain embodiments, the permeation enhancer or combination of permeation enhancers is present in an amount effective to increase the flux of the therapeutic agent across a barrier compared to a reference composition by at least about 2 fold, at least about 2.5 fold, at least about 3 fold, at least about 4 fold, at least about 5 fold, at least about 10 fold, at least about 25 fold, at least about 50 fold, at least about 100 fold, at least about 250 fold, at least about 500 fold, or at least about 1000 fold. In certain embodiments, the permeation enhancer or combination of permeation enhancers is present in an amount effective to increase the flux of the therapeutic agent across a barrier compared to a reference composition by between about 1.5 fold and about 100 fold.

In certain embodiments, the composition comprises one or more types of permeation enhancers. In certain embodiments, the composition comprises one type, two types, or three types of permeation enhancers. In certain embodiments, the composition comprises one or more types of surfactant permeation enhancers, for example, cationic surfactant permeation enhancers (e.g., dodecyltrimethylammonium bromide (DDAB), octyltrimethylammonium bromide (OTAB)), or transdermal chemical permeation enhancers (e.g., 1-dodecylazacycloheptan-2-one, also known as azone or laurocapram). In certain embodiments, the permeation enhancer is the surfactant sodium dodecyl sulfate. In certain embodiments, the permeation enhancer is the anesthetic bupivacaine. In certain embodiments, the permeation enhancer is the terpene limonene. In certain embodiments, the composition comprises the permeation enhancers sodium dodecyl sulfate, limonene, and/or bupivacaine. In certain embodiments, the composition comprises the permeation enhancers sodium dodecyl sulfate, limonene, and bupivacaine. In certain embodiments, the composition comprises the permeation enhancers sodium dodecyl sulfate (“SDS”) and limonene. In certain embodiments, in the composition, the permeation enhancers comprise a surfactant permeation enhancer (e.g., sodium dodecyl sulfate), terpene permeation enhancer (e.g., limonene), and do not include a local anesthetic permeation enhancer (e.g., amino amide local anesthetic, for example, bupivacaine). In certain embodiments, in the composition, the permeation enhancers consist of a surfactant permeation enhancer (e.g., sodium dodecyl sulfate), terpene permeation enhancer (e.g., limonene), and does not include a local anesthetic permeation enhancer (e.g., amino amide local anesthetic, for example, bupivacaine). In certain embodiments, in the composition, the permeation enhancers consist of sodium dodecyl sulfate and limonene. In certain embodiments, the composition comprises the permeation enhancer limonene. In certain embodiments, the permeation enhancer comprises a single permeation enhancer. In certain embodiments, the permeation enhancer comprises two permeation enhancers. In certain embodiments, the permeation enhancer comprises three permeation enhancers. In certain embodiments, the composition comprises between about 0.2-1.0% of sodium dodecyl sulfate, in weight per volume composition (wt/vol). In certain embodiments, the composition comprises between about 0.5-3.2% wt/vol of sodium dodecyl sulfate, for example, between about 0.5-1.5% wt/vol of sodium dodecyl sulfate. In certain embodiments, the composition comprises between about 0.5-3.2% wt/vol of sodium dodecyl sulfate, for example, between about 0.5-1.5% wt/vol, about 1.0-2.7% wt/vol, about 1.8-2.7% wt/vol, about 2.0-2.4% wt/vol, about 2.0-2.6% wt/vol, or about 2.0-3.0% wt/vol of sodium dodecyl sulfate. In certain embodiments, the composition comprises between about 0.2-0.4% wt/vol, about 0.4-0.6% wt/vol, about 0.6-0.8% wt/vol, about 0.8-1.0% wt/vol, about 1.0-1.2% wt/vol, or about 1.2-1.4% wt/vol, of sodium dodecyl sulfate. In certain embodiments, the composition comprises between about 0.2-0.4% wt/vol, about 0.4-0.5% wt/vol, about 0.5-0.6% wt/vol, about 0.6-0.8% wt/vol, about 0.8-1.0% wt/vol, about 1.0-1.2% wt/vol, about 1.2-1.3% wt/vol, about 1.3-1.5% wt/vol, about 1.5-1.6% wt/vol, about 1.6-1.7% wt/vol, about 1.7-1.8% wt/vol, about 1.8-1.9% wt/vol, about 1.9-2.0% wt/vol, about 2.0-2.2% wt/vol, about 2.2-2.4% wt/vol, about 2.4-2.5% wt/vol, about 2.5-2.6% wt/vol, about 2.6-2.7% wt/vol, about 2.7-2.8% wt/vol, about 2.8-2.9% wt/vol, about 2.9-3.0% wt/vol, about 3.0-3.1% wt/vol, about 3.1-3.2% wt/vol, about 3.2-3.3% wt/vol, about 3.3-3.4% wt/vol, or about 3.4-3.5% wt/vol, of sodium dodecyl sulfate. In certain embodiments, the composition comprises between about 0.2-0.4% wt/vol, about 0.4-0.5% wt/vol, about 0.5-0.6% wt/vol, about 0.6-0.8% wt/vol, about 0.8-1.0% wt/vol, about 1.0-1.2% wt/vol, about 1.2-1.3% wt/vol, about 1.3-1.5% wt/vol, about 1.5-1.6% wt/vol, about 1.6-1.7% wt/vol, about 1.7-1.8% wt/vol, about 1.8-1.9% wt/vol, about 1.9-2.0% wt/vol, about 2.0-2.1% wt/vol, about 2.15-2.2% wt/vol, about 2.1-2.2% wt/vol, about 2.0-2.2% wt/vol, about 2.2-2.3% wt/vol, about 2.2-2.4% wt/vol, about 2.3-2.4% wt/vol, about 2.4-2.5% wt/vol, about 2.5-2.6% wt/vol, about 2.6-2.7% wt/vol, about 2.7-2.8% wt/vol, about 2.8-2.9% wt/vol, about 2.9-3.0% wt/vol, about 3.0-3.1% wt/vol, about 3.1-3.2% wt/vol, about 3.2-3.3% wt/vol, about 3.3-3.4% wt/vol, or about 3.4-3.5% wt/vol, of sodium dodecyl sulfate. In certain embodiments, the composition comprises about 1.0% wt/vol of sodium dodecyl sulfate. In certain embodiments, the composition comprises no more than 1.1 wt/vol or 1.2% wt/vol of sodium dodecyl sulfate. In certain embodiments, the composition comprises about 0.2% and 1.5% wt/vol of sodium dodecyl sulfate, between about 0.2-1.0% wt/vol of sodium dodecyl sulfate, between about 0.5-1.5% wt/vol of sodium dodecyl sulfate, or about 1.0% wt/vol of sodium dodecyl sulfate. In certain embodiments, the composition comprises about 1.8% wt/vol, about 1.8% wt/vol, about 2.0% wt/vol, about 2.2% wt/vol, about 2.4% wt/vol, about 2.6% wt/vol, about 2.8% wt/vol, about 3.0% wt/vol, or about 3.2% wt/vol of sodium dodecyl sulfate. In certain embodiments, the composition comprises up to 40% or up to 20% of sodium dodecyl sulfate, in weight per volume composition (wt/vol), for example, between 0.2-20% wt/vol of sodium dodecyl sulfate, between 0.2-10% wt/vol of sodium dodecyl sulfate, or between 0.2-5% wt/vol of sodium dodecyl sulfate. In certain embodiments, the composition comprises between about 0.2-0.4% wt/vol, about 0.4-0.8% wt/vol, about 0.8-1.0% wt/vol, about 0.4-0.5% wt/vol, about 0.5-0.6% wt/vol, about 0.6-0.8% wt/vol, about 0.8-1.0% wt/vol, about 1.0-1.2% wt/vol, about 1.2-1.3% wt/vol, about 1.3-1.5% wt/vol, about 1.5-1.6% wt/vol, about 1.6-1.7% wt/vol, about 1.7-1.8% wt/vol, about 1.8-1.9% wt/vol, about 1.9-2.0% wt/vol, about 2.0-2.1% wt/vol, about 2.15-2.2% wt/vol, about 2.1-2.2% wt/vol, about 2.0-2.2% wt/vol, about 2.2-2.3% wt/vol, about 2.2-2.4% wt/vol, about 2.3-2.4% wt/vol, about 2.4-2.5% wt/vol, about 2.5-2.6% wt/vol, about 2.6-2.7% wt/vol, about 2.7-2.8% wt/vol, about 2.8-2.9% wt/vol, about 2.9-3.0% wt/vol, about 3.0-3.1% wt/vol, about 3.1-3.2% wt/vol, about 3.2-3.3% wt/vol, about 3.3-3.4% wt/vol, or about 3.4-3.5% wt/vol, about 1.0-2.5% wt/vol, about 2.5-5.0% wt/vol, about 5.0-7.0% wt/vol, about 7.0-10.0% wt/vol, about 10.0-12.0% wt/vol, about 12.0-17.0% wt/vol, about 17.0-20.0% wt/vol, about 15.0-20.0% wt/vol, about 22.0-25.0% wt/vol, about 20.0-25.0% wt/vol, about 25.0-27.0% wt/vol, about 27.0-30.0% wt/vol, about 25.0-30.0% wt/vol, about 30.0-32.0% wt/vol, about 32.0-35.0% wt/vol, about 35.0-37.0% wt/vol, about 37.0-40.0% wt/vol, about 40.0-42.0% wt/vol, about 35.0-40.0% wt/vol, about 40.0-45.0% wt/vol, about 45.0-50.0% wt/vol, of sodium dodecyl sulfate. In certain embodiments, the composition comprises up to 40% or up to 20% of sodium dodecyl sulfate, in weight per volume composition (wt/vol), for example, between 0.2-20% wt/vol of sodium dodecyl sulfate. In certain embodiments, the composition comprises the permeation enhancer that is a local anesthetic, for example, an amino amide (e.g., bupivacaine) or amino ester (e.g., tetracaine). In certain embodiments, the composition comprises the permeation enhancer bupivacaine or tetracaine. In certain embodiments, the composition comprises the permeation enhancer bupivacaine, for example, between about 0.2% and 2.0% of the composition by weight per volume composition or between about 0.25% and 0.75% of the composition by weight per volume composition. In certain embodiments, the composition comprises between about 0.0-0.2% wt/vol about 0.2-0.25% wt/vol, about 0.25-0.3% wt/vol, about 0.3-0.4% wt/vol, about 0.4-0.5% wt/vol, about 0.5-0.6% wt/vol, about 0.6-0.7% wt/vol, about 0.7-0.75% wt/vol, about 0.75-0.8% wt/vol, about 0.8-0.9% wt/vol, about 0.9-1.0% wt/vol, about 1.0-1.1% wt/vol, about 1.0% wt/vol, about 1.1-1.2% wt/vol, about 1.2-1.25% wt/vol, about 1.25-1.4% wt/vol, about 1.5-2.0% wt/vol, about 2.0-2.5% wt/vol, about 2.5-3.0% wt/vol, about 3.0-3.5% wt/vol, about 3.5-4.0% wt/vol, about 4.0-4.5% wt/vol, about 4.5-5.0% wt/vol, about 5.0-5.5% wt/vol, about 5.5-6.0% wt/vol, about 6.0-6.5% wt/vol, or about 6.5-7.0% wt/vol, of bupivacaine of the composition by weight per volume composition. In certain embodiments, the composition comprises between about 0.5-1.25% wt/vol, between about 0.3-0.6% wt/vol, about 0.5% wt/vol, about 1.0% wt/vol, about 1.0-1.25% wt/vol, or about 4.0-6.0% wt/vol of limonene. In certain embodiments, the composition comprises the permeation enhancer limonene, for example, in between about 0.2% and 10.0% of the composition by weight per volume composition, between about 0.2-6.0% wt/vol, about 1.0-2.7% wt/vol, about 1.8-2.7% wt/vol, about 2.0-2.6% wt/vol, or between about 2.0% and 6.0% of the composition by wt/vol composition. In certain embodiments, the composition comprises the permeation enhancer limonene, for example, in between about 0.5% and 7.0% of the composition by weight per volume composition, about 0.5-3.5% wt/vol, or about 1.5% and 6.0% of the composition by weight per volume composition. In certain embodiments, the composition comprises about 0.5-3.5% wt/vol, about 2.0% wt/vol, about 2.1% wt/vol, about 2.25% wt/vol, about 2.5% wt/vol, about 3.0% wt/vol, about 3.25% wt/vol, about 3.5% wt/vol, about 3.75% wt/vol, about 4.0% wt/vol, about 4.1% wt/vol, about 4.25% wt/vol, or about 4.5% wt/vol, of limonene. In certain embodiments, the composition comprises between about 0.2-0.25% wt/vol, about 0.25-0.3% wt/vol, about 0.3-0.4% wt/vol, about 0.4-0.5% wt/vol, about 0.5-0.6% wt/vol, about 0.6-0.7% wt/vol, about 0.7-0.75% wt/vol, about 0.75-0.8% wt/vol, about 0.8-0.9% wt/vol, about 0.9-1.0% wt/vol, about 1.0-1.1% wt/vol, about 1.0% wt/vol, about 1.1-1.2% wt/vol, about 1.2-1.25% wt/vol, about 1.25-1.4% wt/vol, about 1.5-1.75% wt/vol, about 1.75-2.0% wt/vol, about 2.0% wt/vol, about 2.0-2.25% wt/vol, about 2.25-2.5% wt/vol, about 2.5-3.0% wt/vol, about 3.0-3.5% wt/vol, about 3.5-4.0% wt/vol, about 4.0-4.25% wt/vol, about 4.25-4.5% wt/vol, about 4.5-4.75% wt/vol, about 4.75-4.8% wt/vol, about 4.8-5.0% wt/vol, about 5.0-5.1% wt/vol, about 5.1-5.2% wt/vol, about 5.2-5.25% wt/vol, or about 5.25-5.5% wt/vol of limonene. In certain embodiments, the composition comprises between about 0.2-0.25% wt/vol, about 0.25-0.3% wt/vol, about 0.3-0.4% wt/vol, about 0.4-0.5% wt/vol, about 0.5-0.6% wt/vol, about 0.6-0.7% wt/vol, about 0.7-0.75% wt/vol, about 0.75-0.8% wt/vol, about 0.8-0.9% wt/vol, about 0.9-1.0% wt/vol, about 1.0-1.1% wt/vol, about 1.0% wt/vol, about 1.1-1.2% wt/vol, about 1.2-1.25% wt/vol, about 1.25-1.4% wt/vol, about 1.5-1.75% wt/vol, about 1.75-2.0% wt/vol, about 2.0% wt/vol, about 2.0-2.25% wt/vol, about 2.25-2.5% wt/vol, about 2.5-3.0% wt/vol, about 3.0-3.5% wt/vol, about 3.5-4.0% wt/vol, about 4.0-4.25% wt/vol, about 4.25-4.5% wt/vol, about 4.5-4.75% wt/vol, about 4.75-4.8% wt/vol, about 4.8-5.0% wt/vol, about 5.0-5.1% wt/vol, about 5.0-5.2% wt/vol, about 5.1-5.2% wt/vol, about 5.2-5.25% wt/vol, or about 5.25-5.5% wt/vol, about 5.5-5.75% wt/vol, about 5.75-6.0% wt/vol, about 6.0-6.25% wt/vol, about 6.25-6.5% wt/vol, about 6.5-6.75% wt/vol, about 6.75-7.0% wt/vol, about 7.0-7.25% wt/vol, about 7.25-7.5% wt/vol, about 7.5-7.75% wt/vol, about 7.75-8.0% wt/vol, about 8.0-8.25% wt/vol, about 8.25-8.5% wt/vol, about 8.5-8.75% wt/vol, about 8.75-9.0% wt/vol, about 9.0-9.25% wt/vol, about 9.25-9.5% wt/vol, about 9.5-9.75% wt/vol, about 9.75-10.0% wt/vol, about 10.0-10.25% wt/vol, about 10.25-10.5% wt/vol, about 10.5-10.75% wt/vol, or about 10.75-11.0% wt/vol, of limonene. In certain embodiments, the composition comprises between about 2.0-6.0% wt/vol, 1.5-10.0% wt/vol, or 1.5-10.5% wt/vol of limonene, for example, about 2.0% wt/vol, about 4.0% wt/vol, about 5.0% wt/vol. In certain embodiments, the composition comprises about 5.0% wt/vol of limonene. In certain embodiments, the composition comprises 10.0% wt/vol or less of limonene. In certain embodiments, the composition comprises up to about 10%, up to about 7%, up to about 6%, or up to about 5% limonene wt/vol.

### Matrix Forming Agent: Block Copolymers

The matrix-forming agent (matrix forming agent) is a compound or mixture of compounds that forms a gel or hydrogel (“gels”) after administration. In certain embodiments, the matrix forming agent forms a gel after administration into a subject's ear canal. The gel composition acts a reservoir containing the therapeutic agent and permeation enhancer, allowing for sustained release of the therapeutic agent across a barrier (e.g., tympanic membrane). In certain embodiments, the gel maintains contact with the tympanic membrane. In some embodiments, the gel maintains contact for between 0.5 and 1 hours, between 1 and 4 hours, between 1 and 8 hours, between 1 and 16 hours, or between 1 and 24 hours. In some embodiments, the gel maintains contact for between 1 day and 3 days, between 1 and 7 days, or between 1 and 14 days. In some embodiments, the gel allows flux of the therapeutic agent across the tympanic membrane for between 0.5 and 1 hours, between 1 and 4 hours, between 1 and 8 hours, between 1 and 16 hours, or between 1 and 24 hours. In some embodiments, the gel maintains contact for between 1 day and 3 days, between 1 and 7 days, or between 1 and 14 days. Such a reservoir maintains contact with the tympanic membrane increasing the time for the therapeutic agent to cross the tympanic membrane and be delivered to the middle or inner ear. Such a reservoir maximizes exposure of the tympanic membrane to permeation enhancers and the therapeutic agent, and facilitates sustained flux of the therapeutic agent into the middle and inner ear.

In various embodiments, the composition is a shelf-stable formulation at room temperature. In certain embodiments, the composition is a sustained release formulation. In various aspects, sustained release of either the permeation enhancer and/or the therapeutic agent can be at a constant rate to deliver an effective amount of either the permeation enhancer or therapeutic agent to the surface of the tympanic membrane, the middle ear, or the inner ear. In various embodiments, the sustained release provides a sufficient flux of therapeutic agent over about 1 day, about 2 days, about 3 days, about 4 days, about 5 days, about 6 days, or about 7 days. In various embodiments, the sustained release provides a sufficient flux of therapeutic agent over a range of about 7 to about 10 days. In various embodiments, the sustained release may be at a constant rate over a range of about 7 days to about 14 days. In various embodiments, the sustained release provides a sufficient flux of therapeutic agent over a range of about 14 to about 21 days. In various embodiments, the sustained release provides a sufficient flux of therapeutic agent over a range of about 21 to about 30 days. As used herein, sufficient flux is the flux necessary for the therapeutic agent to be present in the middle ear in a therapeutically effective amount or prophylactically effective amount. In some embodiments, the sufficient flux is sufficient to provide an antibiotic agent in a concentration equal or greater to the minimum inhibitory concentration of an infectious microorganism. In some embodiments, the infectious microorganism is H. influenza, S. pneumoniae, or M. catarrhalis.

In various aspects, the sustained release profile is obtained by the addition of a matrix-forming agent to the composition. In various embodiments, the composition may further comprise a matrix forming agent. In various embodiments, the matrix forming agents may undergo a change in viscosity, in situ, based on a phase change, a change in solubility, evaporation of a solvent, or mixing of components comprising the matrix forming agent. Such matrix forming agents gel, in situ after administration into a patient's ear canal to form a reservoir containing the therapeutic agent and permeation enhancer, allowing sustained release of the therapeutic agent. Such a reservoir maintains contact with the tympanic membrane increasing the time for the therapeutic agent to permeate the tympanic membrane, and be delivered to the middle or inner ear. Such a reservoir maximizes exposure of the tympanic membrane to permeation enhancers and the therapeutic agent. In certain embodiments, the matrix forming agent comprises a poloxamer (e.g., a copolymer of Formula (I), for example poloxamer P188), another thermosensitive polymer, and/or another non-thermosensitive polymer. In certain embodiments, the matrix forming agent comprises a poloxamer (e.g., a copolymer of Formula (I) as shown below, for example poloxamer P188).

In certain embodiments, the composition comprises a poloxamer, also known as a nonionic triblock copolymer, of Formula (I):

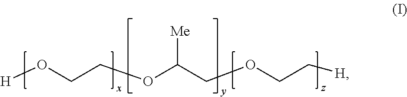

wherein:
x is between 20 and 90;
y is between 25 and 30; and
z is between 20 and 90.

In certain embodiments, the composition comprises a poloxamer of Formula (I), wherein x is between 50-120; y is between 20-35; z is between 50-120; or x is between 70-90; y is between 24-30; and z is between 70-90. In certain embodiments, the composition comprises a poloxamer of Formula (I), wherein x is between 20 and 30, 30 and 40, 40 and 50, 50 and 60, 60 and 70, 70 and 80, 80 and 90, 90 and 100, or 100 and 105; y is between 20 and 25, 25 and 26, 26 and 27, 27 and 28, or 28 and 29; and y is between 20 and 30, 30 and 40, 40 and 50, 50 and 60, 60 and 70, 70 and 80, 80 and 90, 90 and 100, 100 and 105, 105 and 110, or 110 and 115. In certain embodiments, the composition comprises a poloxamer of Formula (I), wherein x is between 80 and 90; y is between 25 and 29; and z is between 80 and 90. In certain embodiments, the composition comprises a poloxamer of Formula (I), wherein x is between 75 and 85; y is between 25 and 28; and z is between 75 and 85. In certain embodiments, the composition comprises a poloxamer of Formula (I), wherein x and y are 80, and z is 27. In certain embodiments, in a poloxamer of Formula (I) in the composition, x and z are each not approximately 2-10 or approximately 130-140; and y is not approximately 23-70. In certain embodiments, the composition comprises a matrix forming agent that is a block copolymer comprising poloxamer P188. In certain embodiments, the composition comprises the poloxamer of Formula (I) that is of poloxamer P188, also known as Kolliphor P188 or P188. In certain embodiments, the composition comprises poloxamer P188, for example, of between about 18% and about 62%, between about 30% and about 50%, between about 30% and about 40%, between about 34% and about 37%, between about 30% and about 31%, between about 31% and about 32%, between about 32% and about 33%, between about 33% and about 34%, between about 34% and about 34.5%, between about 34.5% and about 35%, between about 35% and about 35.5%, between about 35.5% and about 36%, between about 36% and about 36.5%, between about 36.5% and about 37%, between about 37% and about 37.5%, between about 37.5% and about 38%, between about 38% and about 39%, between about 39% and about 40%, or between about 20% and about 50% of the composition by weight per volume composition. In certain embodiments, the composition comprises poloxamer P188, for example, of between about 18-62%, between about 21.0-37.0% wt/vol, between about 30-50%, between about 30-40%, between about 34-37%, between about 20.0-21.5% wt/vol, between about 20.0-21.0% wt/vol, between about 21.0-22.0% wt/vol, between about 20.0-22.0% wt/vol, between about 22.0-23.0% wt/vol, between about 23.0-24.0% wt/vol, between about 22.0-24.0% wt/vol, between about 24.0-25.0% wt/vol, between about 24.0-26.0% wt/vol, between about 25.0-26.0% wt/vol, between about 26.0-27.0% wt/vol, between about 26.0-28.0% wt/vol, between about 27.0-28.0% wt/vol, between about 28.0-29.0% wt/vol, between about 29.0-30.0% wt/vol, between about 28.0-30.0% wt/vol, between about 30-31%, between about 30.0-32.0% wt/vol, between about 32.0-33.0% wt/vol, between about 32.0-34.0% wt/vol, between about 31-32%, between about 32-33%, between about 33-34%, between about 34-34.5%, between about 34.5-35%, between about 35-35.5%, between about 35.5-36%, between about 36-36.5%, between about 36.5-37%, between about 37-37.5%, between about 37.5-38%, between about 38-39%, between about 39-40%, between about 40.0-42.0% wt/vol, between about 42.0-45.0% wt/vol, between about 45.0-47.0% wt/vol, between about 47.0-50.0% wt/vol, between about 50.0-52.0% wt/vol, or between about 20-50% of the composition by weight per volume composition. In certain embodiments, the composition comprises poloxamer P188, for example, of between about 18-62%, between about 21.0-37.0% wt/vol, of the composition by weight per volume composition. In certain embodiments, the composition comprises over 20% wt/vol, over 21% wt/vol, over 22% wt/vol, over 25% wt/vol, over 30% wt/vol, over 32% wt/vol, over 34% wt/vol, over 35% wt/vol, over 36% wt/vol, over 37% wt/vol, or over 40% wt/vol, over 45% wt/vol, of poloxamer P188. In certain embodiments, the composition comprises over 30% wt/vol, over 34% wt/vol, over 35% wt/vol, over 37% wt/vol, or over 40% wt/vol, over 45% wt/vol, of poloxamer P188. In certain embodiments, the composition comprises between about 21.0-37.0% wt/vol (e.g., 21-35%, for example 21%, 23%, 24%, 26%, 35%) of poloxamer P188. In certain embodiments, the composition comprises a percentage of poloxamer P188 as listed in one of the Examples (e.g., in Tables 2 or 4). In certain embodiments, the composition comprises about 21.0% wt/vol, about 23.0% wt/vol, about 24.0% wt/vol, about 25.0% wt/vol, about 26.0% wt/vol, or about 35.0% wt/vol of poloxamer P188. In certain embodiments, the composition comprises about 21.0% wt/vol, about 23.0% wt/vol, about 24.0% wt/vol, about 25.0% wt/vol, about 26.0% wt/vol, about 35.0% wt/vol, about 40.0% wt/vol, about 42.0% wt/vol, or about 45.0% wt/vol of poloxamer P188. In certain embodiments, the composition comprises between about 34.0-37.0% wt/vol of poloxamer P188. In certain embodiments, the poloxamer is poloxamer P182 (Pluronic® L62), poloxamer P331 (Pluronic® L101), poloxamer P124 (Pluronic® L44), poloxamer P401 (Pluronic® L121), poloxamer P181 (Pluronic® L-61), poloxamer P231 (Pluronic® L-81), poloxamer P338 (Pluronic® F-108), and/or Pluronic® 31R1. In certain embodiments, the poloxamer is not poloxamer P407. In certain embodiments, the poloxamer is not poloxamer P407, poloxamer P182 (Pluronic® L62), poloxamer P331 (Pluronic® L101), poloxamer P124 (Pluronic® L44), poloxamer P401 (Pluronic® L121), poloxamer P181 (Pluronic® L-61), poloxamer P231 (Pluronic® L-81), poloxamer P338 (Pluronic® F-108), Pluronic® 31R1, and/or [P407-PBP] which is of the formula

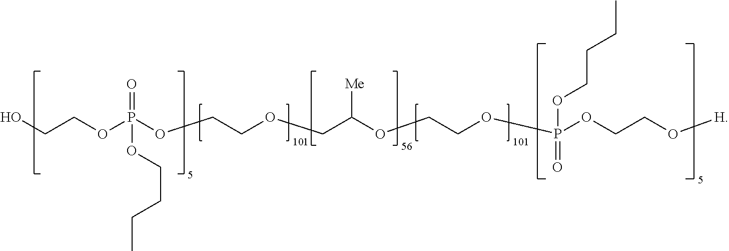

### Phase Transition Temperature

The composition may be a liquid prior to warming above the phase transition temperature. In some embodiments, the composition “forms a gel” or “gels” at temperatures above a phase transition temperature and the phase transition temperature is less than about 37° C., or between about 20° C. and about 37° C. The composition may form a gel when administered to a subject, e.g., when the composition contacts a biological surface. In certain embodiments, the composition is applied to a surface of temperature equal to or above the phase transition temperature. In some embodiments, the surface is a biological surface. In certain embodiments, the surface is skin. In certain embodiments, the surface is a surface in the ear canal of a subject. In certain embodiments, the surface is a tympanic membrane. The composition may be administered to an interior body surface, for example, by intradermal or interdermal delivery or during a surgical procedure. In certain embodiments, the phase transition temperature is between about 24.0° C. and about 37.0° C. In certain embodiments, the phase transition temperature is between about 20.0° C. and about 30.0° C. In certain embodiments, the phase transition temperature is about 25.0° C. In certain embodiments, the phase transition temperature is between about 20° C. and about 22° C., about 22° C. and about 23° C., about 23° C. and about 24° C., about 24° C. and about 25° C., about 25° C. and about 26° C., about 26° C. and about 27° C., about 27° C. and about 29° C., about 29° C. and about 30° C., about 30° C. and about 31° C., about 31° C. and about 32° C., about 32° C. and about 33° C., about 33° C. and about 34° C., about 34° C. and about 34.5° C., about 34.5° C. and about 35° C., about 35° C. and about 35.5° C., about 35.5° C. and about 36° C., about 36° C. and about 36.5° C., about 36.5° C. and about 37° C., or about 37° C. and about 38° C. In certain embodiments, the phase transition temperature is between about 25-35.5° C., about 20-22° C., about 22-23° C., about 23-24° C., about 24-25° C., about 25-26° C., about 25-27° C., about 26-27° C., about 27-29° C., about 29-30° C., about 30-31° C., about 31-32° C., about 32-33° C., about 33-34° C., about 34-34.5° C., about 34.5-35° C., about 35-35.5° C., about 35.5-36° C., about 36-36.5° C., about 36.5-37° C., or about 37-38° C. In certain embodiments, the phase transition temperature is about 25.0° C., about 27.0° C., about 30.0° C., about 31.0° C., about 33.0° C., about 34.0° C., or about 35.5° C. In certain embodiments, the phase transition temperature is between about 29.0° C. and about 36.5° C. or between about 34.0° C. and about 36.5° C. As described, the gelation temperature (phase transition temperature) of the composition is one factor in determining whether the suitability of the composition (e.g., to allow for sustained delivery to the tympanic membrane). The temperature at which the storage modulus exceeds the loss modulus is considered the gelation temperature. Compositions herein may have a gelation temperature preferably lower than 37° C. to accelerate gelation right after administration upon exposure of the composition, in particular the matrix forming agent, to body heat.

The timing of the sol-gel transition will impact the ease of administration. In general a faster in situ transition is useful for administration to subjects (e.g., children resisting compliance). In certain embodiments, the composition gels within about 5 seconds (“s”), about 10 s, about 12 s, about 20 s, about 28 s, about 30 s, about 30-35 s, about 1 minute, about 5 minutes, or about 10 minutes of administration (e.g., to the ear canal). In some embodiments, the composition gels in the range of about 1 s to about 20 s after administration. In some embodiments, the composition gels in the range of about 10 s to about 1 minute after administration.

In certain embodiments, the composition is stored cold (e.g., refrigerated at about 5° C.) prior to administration. Cold storage may be useful for compositions with gelation temperatures below room temperature to prevent gelation prior to administration or during handling.

### Therapeutic Agents

A therapeutic agent can be any agent used to treat any ear disease, or symptom of an ear disease or infectious disease (e.g., pain associated with an ear disease or infectious disease). A therapeutic agent can be an agent used to treat pain. Therapeutic agents may include antimicrobial agents. Therapeutic agents may include, but are not limited to, antimicrobial agents, antibiotics, anesthetics, anti-inflammatories, analgesics, anti-fibrotics, anti-sclerotics, and anticoagulants. Therapeutic agents may include, but are not limited to, antibiotics, anesthetics, anti-inflammatories, analgesics, anti-fibrotics, anti-sclerotics, and anticoagulants. In certain embodiments, the therapeutic agent is an antimicrobial agent. In certain embodiments, the therapeutic agent is an antibiotic agent. In certain embodiments, the therapeutic agent is an anesthetic agent. In certain embodiments, the therapeutic agent is an anti-inflammatory agent. In certain embodiments, the therapeutic agent is an analgesic agent. In certain embodiments, the therapeutic agent is an anti-fibrotic agent. In certain embodiments, the therapeutic agent is an anti-sclerotic agent. In certain embodiments, the therapeutic agent is an anticoagulant agent. In certain embodiments, the therapeutic agent is present as a pharmaceutically acceptable salt or a free base of the active agent. In certain embodiments, the therapeutic agent is present as a pharmaceutically acceptable salt of the active agent.

In various aspects, the therapeutic agents may comprise between about 0.01 percent to about 10 percent of the composition. In various embodiments, the therapeutic agents may comprise between about 0.01 percent to about 1 percent of the composition, comprise between about 1 percent to about 2 percent of the composition, comprise between about 2 percent to about 3 percent of the composition, comprise between about 3 percent to about 4 percent of the composition, comprise between about 4 percent to about 5 percent of the composition, comprise between about 5 percent to about 6 percent of the composition, comprise between about 6 percent to about 7 percent of the composition, comprise between about 7 percent to about 8 percent of the composition, comprise between about 8 percent to about 9 percent of the composition, or comprise between about 9 percent to about 10 percent of the composition.

In various aspects, the therapeutic agents may comprise between about 0.01 percent to about 10 percent wt/vol of the composition. In various aspects, the therapeutic agents may comprise between about 1.0 percent to about 7.0 percent wt/vol of the composition. In various aspects, the therapeutic agents may comprise between about 1.0 percent to about 6.0 percent wt/vol of the composition.

The exact amount required will vary from subject to subject, depending on the species, age, and general condition of the subject, the particular compound, its mode of administration, its mode of activity, condition being treated, and the like. The compositions described herein are preferably formulated in dosage unit form for ease of administration and uniformity of dosage. It will be understood, however, that the total daily usage of the compounds and compositions will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, the therapeutic agent is an antimicrobial agent. In certain embodiments, the therapeutic agent is an antibiotic. Any antibiotic may be used in the system. In certain embodiments the antibiotic is approved for use in humans or other animals. In certain embodiments the antibiotic is approved for use by the U.S. Food & Drug Administration. In certain embodiments, the antibiotic may be selected from the group consisting of cephalosporins, quinolones, polypeptides, macrolides, penicillins, and sulfonamides. Exemplary antibiotics may include, but are not limited to, ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole.

In certain embodiments, the therapeutic agent is an antibiotic agent, anesthetic agent, anti-inflammatory agent, analgesic agent, anti-fibrotic agent, anti-sclerotic agent, anticoagulant agent, or diagnostic agent. In certain embodiments, the antibiotic is a quinolone, for example, a fluoroquinolone. In certain embodiments, the antibiotic is a carbapenem. In certain embodiments, the antibiotic is a quinolone (e.g., fluoroquinolone) or a beta lactam antibiotic (e.g., penicillin, cephalosporin (e.g., ceftriaxone)). In certain embodiments, the antibiotic is amoxicillin, azithromicicn, cefuroxime, ceftriaxone, trimethoprim, levofloxacin, moxifloxacin, meropenem, or ciprofloxacin. In some embodiments, the antibiotic is ciprofloxacin. In some embodiments, the antibiotic is ciprofloxacin and pharmaceutically acceptable salts thereof. In some embodiments, the antibiotic is ciprofloxacin hydrochloride. In some embodiments, the antibiotic is levofloxacin. In some embodiments, the antibiotic is ceftriaxone. In some embodiments, the antibiotic is ciprofloxacin, gatifloxacin, ceftriaxone, gemifloxacin, moxalactam, levofloxacin, meropenem, or ampicillin; or pharmaceutically acceptable salts thereof. In some embodiments, the antibiotic is ciprofloxacin, gatifloxacin, ceftriaxone, gemifloxacin, moxalactam, levofloxacin, meropenem, or ampicillin. In some embodiments, the antibiotic (e.g., fluoroquinolone or a beta lactam antibiotic, for example, ciprofloxacin, ceftriaxone) is formulated in the composition from a powder form of the antibiotic. In some embodiments, the antibiotic (e.g., fluoroquinolone or a beta lactam antibiotic, for example, ciprofloxacin, ceftriaxone) is formulated in the composition from a liquid form of the antibiotic.

Exemplary antibiotics, include, but are not limited to: Abamectin, Actinomycin (e.g., Actinomycin A, Actinomycin C, Actinomycin D, Aurantin), Alatrofloxacin mesylate, Amikacin sulfate, Amino salicylic acid, Anthracyclines (e.g., Aclarubicin, Adriamycin, Doxorubicin, Epirubicin, Idarubicin), Antimycin (e.g., Antimycin A), Avermectin, BAL 30072, Bacitracin, Bleomycin, Cephalosporins (e.g., 7-Aminocephalosporanic acid, 7-Aminodeacetoxycephalo sporanic acid, Cefaclor, Cefadroxil, Cefamandole, Cefazolin, Cefepime, Cefixime, Cefmenoxime, Cefmetazole, Cefoperazone, Cefotaxime, Cefotetan, Cefotiam, Cefoxitin, Cefpirome, Cefpodoxime proxetil, Cefsulodin, Cefsulodin sodium, Ceftazidime, Ceftizoxime, Ceftriaxone, Cefuroxime, Cephalexin, Cephaloridine, Cephalosporin C, Cephalothin, Cephalothin sodium, Cephapirin, Cephradine), Ciprofloxacin, Enrofloxacin, Clarithromycin, Clavulanic acid, Clindamycin, Colicin, Cyclosporin (e.g. Cyclosporin A), Dalfopristin/quinupristin, Daunorubicin, Doxorubicin, Epirubicin, GSK 1322322, Geneticin, Gentamicin, Gentamicin sulfate, Gramicidin (e.g. Gramicidin A), Grepafloxacin hydrochloride, Ivermectin, Kanamycin (e.g. Kanamycin A), Lasalocid, Leucomycin, Levofloxacin, Linezolid, Lomefloxacin, Lovastatin, MK 7655, Meropenem, Mevastatin, Mithramycin, Mitomycin, Monomycin, Natamycin, Neocarzinostatin, Neomycin (e.g. Neomycin sulfate), Nystatin, Oligomycin, Olivomycin, Pefloxacin, Penicillin (e.g. 6-Aminopenicillanic acid, Amoxicillin, Amoxicillin-clavulanic acid, Ampicillin, Ampicillin sodium, Azlocillin, Carbenicillin, Cefoxitin, Cephaloridine, Cloxacillin, Dicloxacillin, Mecillinam, Methicillin, Mezlocillin, Nafcillin, Oxacillin, Penicillin G, Penicillin G potassium, Penicillin G procaine, Penicillin G sodium, Penicillin V, Piperacillin, Piperacillin-tazobactam, Sulbactam, Tazobactam, Ticarcillin), Phleomycin, Polymyxin (e.g., Colistin, Polymyxin B), Pyocin (e.g. Pyocin R), RPX 7009, Rapamycin, Ristocetin, Salinomycin, Sparfloxacin, Spectinomycin, Spiramycin, Streptogramin, Streptovaricin, Tedizolid phosphate, Teicoplanin, Telithromycin, Tetracyclines (e.g. Achromycin V, Demeclocycline, Doxycycline, Doxycycline monohydrate, Minocycline, Oxytetracycline, Oxytetracycline hydrochloride Tetracycline, Tetracycline hydrochloride), Trichostatin A, Trovafloxacin, Tunicamycin, Tyrocidine, Valinomycin, (−)-Florfenicol, Acetylsulfisoxazole, Actinonin, Amikacin sulfate, Benzethonium chloride, Cetrimide, Chelerythrine, Chlorhexidine (e.g., Chlorhexidine gluconate), Chlorhexidine acetate, Chlorhexidine gluconate, Chlorothalonil, Co-Trimoxazole, Dichlorophene, Didecyldimethylammonium chloride, Dihydrostreptomycin, Enoxacin, Ethambutol, Fleroxacin, Furazolidone, Methylisothiazolinone, Monolaurin, Oxolinic acid, Povidone-iodine, Spirocheticides (e.g., Arsphenamine, Neoarsphenamine), Sulfaquinoxaline, Thiamphenicol, Tinidazole, Triclosan, Trovafloxacin, Tuberculostatics (e.g., 4-Aminosalicylic acid, AZD 5847, Aminosalicylic acid, Ethionamide), Vidarabine, Zinc pyrithione, and Zirconium phosphate.

In certain embodiments, the therapeutic agent is a Food and Drug Administration (FDA) approved drug for treating infections or infectious diseases. Exemplary FDA approved agents include, but are not limited to: Avycaz (ceftazidime-avibactam), Cresemba (isavuconazonium sulfate), Evotaz (atazanavir and cobicistat, Prezcobix (darunavir and cobicistat), Dalvance (dalbavancin), Harvoni (ledipasvir and sofosbuvir), Impavido (miltefosine), Jublia (efinaconazole), Kerydin (tavaborole), Metronidazole, Orbactiv (oritavancin), Rapivab (peramivir injection), Sivextro (tedizolid phosphate), Triumeq (abacavir, dolutegravir, and lamivudine), Viekira Pak (ombitasvir, paritaprevir, ritonavir and dasabuvir), Xtoro (finafloxacin), Zerbaxa (ceftolozane+tazobactam), Luzu (luliconazole), Olysio (simeprevir), Sitavig (acyclovir), Sovaldi (sofosbuvir), Abthrax (raxibacumab), Afinitor (everolimus), Cystaran (cysteamine hydrochloride), Dymista (azelastine hydrochloride and fluticasone propionate), Fulyzaq (crofelemer), Jetrea (ocriplasmin), Linzess (linaclotide), Qnasl (beclomethasone dipropionate) nasal aerosol, Sirturo (bedaquiline), Sklice (ivermectin), Stribild (elvitegravir, cobicistat, emtricitabine, tenofovir disoproxil fumarate), Tudorza Pressair (aclidinium bromide inhalation powder), Complera (emtricitabine/rilpivirine/tenofovir disoproxil fumarate), Dificid (fidaxomicin), Edurant (rilpivirine), Eylea (aflibercept), Firazyr (icatibant), Gralise (gabapentin), Incivek (telaprevir), Victrelis (boceprevir), Egrifta (tesamorelin), Teflaro (ceftaroline fosamil), Zymaxid (gatifloxacin), Bepreve (bepotastine besilate), Vibativ (telavancin), Aptivus (tipranavir), Astepro (azelastine hydrochloride nasal spray), Intelence (etravirine), Patanase (olopatadine hydrochloride), Viread (tenofovir disoproxil fumarate), Isentress (raltegravir), Selzentry (maraviroc), Veramyst (fluticasone furoate), Xyzal (levocetirizine dihydrochloride), Eraxis (anidulafungin), Noxafil (posaconazole), Prezista (darunavir), Tyzeka (telbivudine), Veregen (kunecatechins), Baraclude (entecavir), Fuzeon (enfuvirtide), Lexiva (fosamprenavir calcium), Reyataz (atazanavir sulfate), Clarinex, Hepsera (adefovir dipivoxil), Pegasys (peginterferon alfa-2a), Sustiva, Vfend (voriconazole), Zelnorm (tegaserod maleate), Avelox (moxifloxacin hydrochloride), Cancidas, Invanz, Peg-Intron (peginterferon alfa-2b), Rebetol (ribavirin), Spectracef, Tavist (clemastine fumarate), Twinrix, Valcyte (valganciclovir HCl), Xigris (drotrecogin alfa), ABREVA (docosanol), Cefazolin, Kaletra, Lamisil (terbinafine hydrochloride), Lotrisone (clotrimazole/betamethasone diproprionate), Lotronex (alosetron HCL), Trizivir (abacavir sulfate, lamivudine, zidovudine AZT), Synercid, Synagis, Viroptic, Aldara (imiquimod), Bactroban, Ceftin (cefuroxime axetil), Combivir, Condylox (pokofilox), Famvir (famciclovir), Floxin, Fortovase, INFERGEN (interferon alfacon-1), Intron A (interferon alfa-2b, recombinant), Mentax (butenafine HCl), Norvir (ritonavir), Omnicef, Rescriptor (delavirdine mesylate), Taxol, Timentin, Trovan, VIRACEPT (nelfinavir mesylate), Zerit (stavudine), AK-Con-A (naphazoline ophthalmic), Allegra (fexofenadine hydrochloride), Astelin nasal spray, Atrovent (ipratropium bromide), Augmentin (amoxicillin/clavulanate), Crixivan (Indinavir sulfate), Elmiron (pentosan polysulfate sodium), Havrix, Leukine (sargramostim), Merrem (meropenem), Nasacort AQ (triamcinolone acetonide), Tavist (clemastine fumarate), Vancenase AQ, Videx (didanosine), Viramune (nevirapine), Zithromax (azithromycin), Cedax (ceftibuten), Clarithromycin (Biaxin), Epivir (lamivudine), Invirase (saquinavir), Valtrex (valacyclovir HCl), Zyrtec (cetirizine HCl), Acyclovir, Penicillin (penicillin g potassium), Cubicin (Daptomycin), Factive (Gemifloxacin), Albenza (albendazole), Alinia (nitazoxanide), Altabax (retapamulin), AzaSite (azithromycin), Besivance (besifloxacin ophthalmic suspension), Biaxin XL (clarithromycin extended-release), Cayston (aztreonam), Cleocin (clindamycin phosphate), Doribax (doripenem), Dynabac, Flagyl ER, Ketek (telithromycin), Moxatag (amoxicillin), Rapamune (sirolimus), Restasis (cyclosporine), Tindamax (tinidazole), Tygacil (tigecycline), and Xifaxan (rifaximin). In certain embodiments, the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In certain embodiments, the antibiotic agent is ciprofloxacin, gatifloxacin, ceftriaxone, gemifloxacin, moxalactam, levofloxacin, meropenem, or ampicillin. In certain embodiments, the antibiotic agent is ciprofloxacin or ceftriaxone. In certain embodiments, the antibiotic agent is ciprofloxacin. In certain embodiments, the antibiotic agent is ceftriaxone. In certain embodiments, the composition comprises between about 1.0-5.0% wt/vol, between about 2.0-5.0% wt/vol, about 1.0-2.0% wt/vol, about 2.0-3.0% wt/vol, about 3.0-4.0% wt/vol, about 4.0-5.0% wt/vol, or about 5.0-6.0% wt/vol, about 5.0-7.0% wt/vol, about 7-8% wt/vol, about 8-9% wt/vol, about 10-12% wt/vol, about 12-14% wt/vol, about 14-15% wt/vol, about 15-17% wt/vol, about 17-18% wt/vol, about 18-19% wt/vol, about 17-19% wt/vol, about 19-20% wt/vol, about 20-21% wt/vol, about 21-22% wt/vol, about 20-22% wt/vol, about 22-24% wt/vol, about 24-25% wt/vol, about 25-26% wt/vol, about 24-26% wt/vol, about 26-27% wt/vol, about 27-28% wt/vol, about 26-28% wt/vol, about 28-29% wt/vol, about 29-30% wt/vol, about 28-30% wt/vol, about 30-31% wt/vol, about 31-32% wt/vol, about 30-32% wt/vol, about 32-33% wt/vol, about 32-34% wt/vol, about 34-36% wt/vol, about 36-38% wt/vol, about 38-40% wt/vol, about 40-42% wt/vol, about 41-42% wt/vol, about 42-43% wt/vol, about 43-44% wt/vol, about 45-46% wt/vol, about 46-47% wt/vol, about 47-48% wt/vol, about 48-49% wt/vol, or about 49-50% wt/vol, of antibiotic (e.g., ciprofloxacin, ceftriaxone). In certain embodiments, the composition comprises between about 3.0-50.0% wt/vol (e.g., about 4-45% wt/vol, for example 4%, 17%, 20%, 21%, 22%, 25%, 28%, 30%, 32%, 35%, 40%, 43%, 45%) of the antibiotic agent (e.g., ciprofloxacin, ceftriaxone). In certain embodiments, the composition comprises between about 3.0-50.0% wt/vol (e.g., about 4-45% wt/vol, for example 4%, 17%, 20%, 21%, 22%, 25%, 28%, 30%, 32%, 35%, 40%, 43%, 45%) of the antibiotic agent that is ciprofloxacin or ceftriaxone. In certain embodiments, the composition comprises between about 1.0-5.0% wt/vol, between about 2.0-5.0% wt/vol, about 1.0-2.0% wt/vol, about 2.0-3.0% wt/vol, about 3.0-4.0% wt/vol, about 4.0-5.0% wt/vol, or about 5.0-6.0% wt/vol, of ciprofloxacin. In certain embodiments, the composition comprises between about 1.0-5.0% wt/vol of ciprofloxacin.

In certain embodiments, the composition comprises the following combination (A):
about 1.0% wt/vol of sodium dodecyl sulfate;
about 0.5% wt/vol of bupivacaine;
about 2.0% wt/vol of limonene; and
about 45.0% wt/vol of poloxamer P188.
   In certain embodiments, the composition comprises the following combination (B):
about 0.4-1.0% wt/vol of sodium dodecyl sulfate;
about 0.5% wt/vol of bupivacaine;
about 2.0% wt/vol of limonene; and
about 45.0% wt/vol of poloxamer P188.
   In certain embodiments, the composition comprises the following combination (C):
about 0.4-1.0% wt/vol of sodium dodecyl sulfate;
about 0.5% wt/vol of bupivacaine;
about 2.0% wt/vol of limonene;
about 45.0% wt/vol of poloxamer P188; and
about 2.0-5.0% wt/vol of ciprofloxacin.
   In certain embodiments, the composition comprises the following combination (D):
about 1.0% wt/vol of sodium dodecyl sulfate;
about 0.5% wt/vol of bupivacaine;
about 2.0% wt/vol of limonene;
about 45.0% wt/vol of poloxamer P188; and
about 2.0-5.0% wt/vol of ciprofloxacin.
   In certain embodiments, the composition comprises the following combination (D):
about 4.0-6.0% wt/vol of limonene;
about 45.0% wt/vol of poloxamer P188; and
about 2.0-5.0% wt/vol of ciprofloxacin.
   In certain embodiments, the composition comprises the following combination (E):
about 0.2-1.0% wt/vol of sodium dodecyl sulfate;
about 2.0% wt/vol of limonene;
about 45.0% wt/vol of poloxamer P188; and
about 2.0-5.0% wt/vol of ciprofloxacin.
   In certain embodiments, the composition comprises the following combination (1E):
about 1.0% wt/vol of sodium dodecyl sulfate;
about 0.5% wt/vol of bupivacaine;
about 2.0% wt/vol of limonene; and
about 45.0% wt/vol of poloxamer P188; and
about 2.0-5.0% wt/vol of therapeutic agent (e.g., antibiotic).
   In certain embodiments, the composition comprises the following combination (1E-1):
about 1.5-3.2% wt/vol of sodium dodecyl sulfate;
about 2.0-5.0% wt/vol of limonene;
about 21.0-37.0% wt/vol of poloxamer P188; and
about 4.0-45.0% wt/vol of therapeutic agent (e.g., antibiotic).
   In certain embodiments, the composition comprises the following combination (1E-2):
about 2.0-3.1% wt/vol of sodium dodecyl sulfate;
about 4.0-5.0% wt/vol of limonene;
about 21.0-36.0% wt/vol of poloxamer P188; and
about 16.0-43.0% wt/vol of therapeutic agent (e.g., antibiotic).
   In certain embodiments, the composition comprises the following combination (1E-3):
about 2.0-3.1% wt/vol of sodium dodecyl sulfate;
about 4.0-5.0% wt/vol of limonene;
about 21.0-36.0% wt/vol of poloxamer P188; and
about 16.0-43.0% wt/vol of ceftriaxone.
   In certain embodiments, the composition comprises the following combination (2E):
about 1.0% wt/vol of sodium dodecyl sulfate;
about 2.0% wt/vol of limonene; and
about 45.0% wt/vol of poloxamer P188; and
about 2.0-5.0% wt/vol of therapeutic agent (e.g., antibiotic).
   In certain embodiments, the composition comprises the following combination (F):
about 1.5-3.2% wt/vol of sodium dodecyl sulfate;
about 3.0-5.0% wt/vol of limonene;
about 34.0-37.0% wt/vol of poloxamer P188; and
about 2.0-5.0% wt/vol of antibiotic.
   In certain embodiments, the composition comprises the following combination (G):
between about 0.4-41% wt/vol of sodium dodecyl sulfate (e.g., about 1.0-40% wt/vol);
about 1.5-10.0% wt/vol of limonene (e.g., up to 40% wt/vol);
about 21.0-37.0% wt/vol of poloxamer P188; and
about 4.0-45.0% wt/vol of therapeutic agent (e.g., antibiotic). In certain embodiments, the composition comprises the combination (A), (B), (C), (D), (E), (1E), (2E), (F), and/or a formulation disclosed in Examples 1-4 (e.g., formulations 1-6 in Example 4). In certain embodiments, the composition comprises the combination (A), (B), (C), (D), (E), (1E), (2E), (F), and/or a formulation disclosed in Examples 1-4 (e.g., formulations 1-6 in Example 4). In certain embodiments, the composition comprises the combination (A), (B), (C), (D), (E), (1E), (2E), and/or (F), and does not comprise fruit oil (e.g., passion fruit oil). In certain embodiments, the composition comprises, a formulation disclosed in Examples 1-4 (e.g., formulations 1-6 in Example 4).
   In certain embodiments, the composition comprises the combination (A), (B), (C), (D), (E), (1E), (1E-1), (1E-2), (1E-3), (2E), (F), and/or a formulation disclosed in Examples 1-6 (e.g., formulations 1-6 in Example 4; formulations 1-11 in Example 6). In certain embodiments, the composition comprises the combination (A), (B), (C), (D), (E), (1E), (1E-1), (1E-2), (1E-3), (2E), (F), (G), and/or a formulation disclosed in Examples 1-6 (e.g., formulations 1-6 in Example 4; formulations 1-11 in Example 6). In certain embodiments, the composition comprises the combination (A), (B), (C), (D), (E), (1E), (1E-1), (1E-2), (1E-3), (2E), (F), and/or (G), and does not comprise fruit oil (e.g., passion fruit oil). In certain embodiments, the composition comprises, a formulation disclosed in Examples 1-6 (e.g., formulations 1-6 in Example 4; formulations 1-11 in Example 6).

### Other Additives

In certain embodiments, the composition comprises one or more additional additives. For example, an additional additive may be a diluent, binding agent, preservative, buffering agent, lubricating agent, perfuming agent, antiseptic agent, or oil. In certain embodiments, the composition does not comprise fruit or vegetable oil (e.g., passion fruit oil).

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent. In certain embodiments, the preservative is benzalkonium chloride.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof.

The composition may comprise water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof.

Formulations suitable for administration (e.g., to the ear canal) include, but are not limited to, liquid and/or semi-liquid preparations such as liniments, lotions, oil-in-water, and/or water-in-oil emulsions such as creams, ointments, and/or pastes, and/or solutions and/or suspensions. Topically administrable formulations may, for example, comprise from about 1% to about 10% (w/w) therapeutic agent, although the concentration of the therapeutic agent can be as high as the solubility limit of the active ingredient in the solvent.

### Pharmaceutical Compositions

In another aspect, provided herein are pharmaceutical compositions comprising at least one of the compositions as described herein, and optionally a pharmaceutically acceptable excipient. In certain embodiments, the pharmaceutical composition includes a combination of therapeutic agents. In certain embodiments, the pharmaceutical composition includes an antibiotic and an additional therapeutic agent. In certain embodiments, the pharmaceutical composition includes an antibiotic agent and an anti-inflammatory agent. In certain embodiments, the pharmaceutical composition includes more than one antibiotic agent. In certain embodiments, the pharmaceutical composition comprises an effective amount (e.g., therapeutically effective amount) of the composition for use in treating a disease in a subject in need thereof.

The disease being treated by the pharmaceutical composition described herein, in certain embodiments, is an infectious disease (e.g., bacterial infection, for example, an H. influenzae, S. pneumoniae, or M. catarrhalis infection; otitis media) and/or an ear disease. In certain embodiments, the additional therapeutic agent is an anti-inflammatory agent (e.g., a steroid). In certain embodiments, the first therapeutic agent is an antibiotic and the additional therapeutic agent is an anti-inflammatory agent. In certain embodiments, the first therapeutic agent is an antibiotic and the additional therapeutic agent is a steroid. Steroids include, but are not limited to, cortisol, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, ciclesonide, and prednicarbate. In some embodiments, the additional anti-inflammatory agent is dexamethasone.

In certain embodiments, the additional therapeutic agent is a β-lactamase inhibitor. In certain embodiments, the first therapeutic agent is an antibiotic (e.g., a β-lactam) and the additional therapeutic agent is a β-lactamase inhibitor. β-Lactamase inhibitors include, but are not limited to, avibactam, clavulanic acid, tazobactam, and sulbactam. The β-lactamase inhibitor may be particularly useful in compositions comprising a β-lactam antibiotic. The β-lactamase inhibitor may increase the efficacy of a β-lactam antibiotic or allow for the β-lactam antibiotic to be present in the composition in a lower concentration than for compositions not containing a β-lactamase inhibitor.

Furthermore, after formulation with an appropriate pharmaceutically acceptable carrier in a desired dosage, the pharmaceutical compositions can be administered to humans and/or other animals. Dosage forms include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the compositions can also include adjuvants such as wetting agents, emulsifying and suspending agents, and perfuming agents. In certain embodiments, the composition comprises a solubilizing agents such an Cremophor, alcohols, oils, modified oils, glycols, polysorbates, cyclodextrins, polymers, and combinations thereof.

It will also be appreciated that the compositions described herein can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound or composition disclosed herein may be administered concurrently with another anticancer agent), or they may achieve different effects (e.g., control of any adverse effects).

In certain embodiments, the composition comprises a diagnostic agent. In some embodiments, the diagnostic agent is an X-ray contrast agent. In some embodiments, the diagnostic agent comprises a radioactive isotope. In some embodiments, the diagnostic agent is a dye.

### Methods of Treatment and Uses

Provided herein are methods and/or uses of the compositions described herein for treating a disease or condition in a subject in need thereof. Methods of using the various embodiments of the compositions described herein are generally directed to methods of treating an infectious disease and/or an ear disease. In certain embodiments, the compositions described herein are used in a method of treating an infectious disease. In certain embodiments, the compositions described herein are used in a method of treating an ear disease. In certain embodiments, the compositions described herein are used in a method of treating an infectious ear disease. In various aspects, the compositions may be used to deliver therapeutic or diagnostic agents across the tympanic membrane. Therefore, the compositions are particularly useful in treating diseases and/or conditions of the middle and/or inner ear. In certain embodiments, the compositions described herein are used in a method of treating diseases and/or conditions of the middle ear. In certain embodiments, the compositions described herein are used in a method of treating diseases and/or conditions of the inner ear. In another aspect, provided are uses of a composition described herein, to treat and/or prevent a disease or condition (e.g., an infectious disease, ear disease, bacterial infection) in a subject in need thereof, the use comprising administering to the subject a therapeutically effective amount of a composition or pharmaceutical composition described herein.

In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

In various aspects, compositions described herein can be used to treat ear diseases, including, but not limited to, ear infections, development of fibroids in the middle ear, or otosclerosis. In certain embodiments, the matrix forming agents described herein can be used to treat ear diseases, including, but not limited to, ear infections, development of fibroids in the middle ear, or otosclerosis. In various other aspects, compositions described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In various other aspects, compositions described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In certain embodiments, the matrix forming agents described herein may be used may treat vertigo, Meniere's disease, mastoiditis, cholesteatoma, labyrinthitis, perilymph fistula, superior canal dehiscence syndrome, otorrhea, otalgia, tinnitus, barotrauma, cancers of the ear, autoimmune inner ear disease acoustic neuroma, benign paroxysmal positional vertigo, herpes zoster oticus, purulent labyrinthitis, vestibular neuronitis, eardrum perforation, or myringitis. In some embodiments, the methods disclosed herein are used for treating otitis media (OM). Different forms of OM, which may be treated by the methods disclosed herein, may be differentiated by the presence of fluid (effusion) and/or by the duration or persistence of inflammation. In certain embodiments, the infectious disease is acute otitis media, chronic otitis media, or secretory otitis media. Effusions, if present, can be of any consistency, from water-like (serous) to viscid and mucous-like (mucoid), to pus-like (purulent); duration is classified as acute, subacute, or chronic. OM with effusion (OME) indicates inflammation with middle ear fluid (MEF), but in the absence of any indications of acute infection. Acute OM (AOM), with or without effusion, is characterized by rapid onset of the signs and symptoms associated with acute infection in the middle ear (e.g., otalgia, fever). In some embodiments, the methods are used for treating otitis media associated with infection by any of a number of pathogenic bacteria, including, for example, Haemophilus influenzae (H. influenzae), Streptococcus pneumoniae (S. pneumoniae), and/or Moraxella catarrhalis (M. catarrhalis). In certain embodiments, treating otitis media comprises clearing the infection by the pathogenic bacteria, including, for example, Haemophilus influenzae (H. influenzae), Streptococcus pneumoniae (S. pneumoniae), and/or Moraxella catarrhalis (M. catarrhalis). In certain embodiments, treating otitis media comprises clearing the infection by H. influenzae.

The infectious disease may be a bacterial infection. In certain embodiments, the bacterial infection is a Streptococcus, Haemophilus, or Moraxella infection. In certain embodiments, the bacterial infection is an H. influenzae, S. pneumoniae, or M. catarrhalis infection. In certain embodiments, the bacterial infection is an infection of the middle ear, for example, an infection with H. influenzae, S. pneumoniae, and/or M. catarrhalis. In certain embodiments, the bacterial infection is a Staphylococcus, Escherichia, or Bacillus infection. In certain embodiments, the bacterial infection is an H. influenzae infection. In certain embodiments, the bacterial infection is a S. pneumoniae infection. In certain embodiments, the bacterial infection is an M. catarrhalis infection. In certain embodiments, the infectious disease is an ear infection. In certain embodiments, the infectious disease is otitis media.

In some embodiments, provided are methods of delivering a composition described herein, the method comprising administering the composition to an ear canal of a subject, for example, wherein the composition contacts the surface of a tympanic membrane. In various embodiments, administration of the compositions described herein comprises applying the composition into a subject's ear canal. In certain embodiments, applying the composition into a subject's ear canal comprises spraying the composition into a subject's ear canal. In certain embodiments, administering the composition to the ear canal comprises placing drops of the composition into the ear canal (e.g., using an applicator (e.g., syringe, catheter) to place the composition into the ear canal, placing the composition into the ear canal with a syringe), or placing a dose of the composition into the ear canal using a catheter. In certain embodiments, administration of the compositions described herein comprises applying the composition into the inner ear of a subject. In certain embodiments, administration of the compositions described herein comprises applying the composition into the middle ear of a subject. In some embodiments, provided are methods of delivering a composition described herein, to the middle ear and/or inner ear of a subject. In some embodiments, provided are methods of delivering a composition described herein, to the tympanic membrane of a subject. In certain embodiments, administration of the compositions described herein comprises applying the composition into the inner ear, sinuses, the eye, or skin of a subject. In certain embodiments, administration of the compositions described herein comprises applying the composition into the sinuses of a subject. In certain embodiments, administration of the compositions described herein comprises applying the composition into the eye of a subject. In certain embodiments, administration of the compositions described herein comprises applying the composition to the skin of a subject. In some embodiments, the drops are delivered from a dropper (e.g., pipet, eye dropper). In some embodiments, the drops are delivered by a syringe. The syringe may be attached to a needle, rigid catheter, or flexible catheter. In certain embodiments, the method of delivering comprises administering the composition on the round window membrane to deliver the composition to the inner ear. In certain embodiments, the method of delivering comprises placing a dose of the composition into the ear canal using a catheter. In some embodiments the catheter is attached to a syringe. In some embodiments, the catheter is rigid. In some embodiments the catheter is flexible. In certain embodiments, the method of delivering comprises placing a dose of the composition into the ear canal using a needle. In some embodiments, the needle is attached to a syringe. In some embodiments, the needle has a blunt tip. In certain embodiments, the method of delivering comprises placing a dose of the composition into the ear canal using a double barrel syringe. The double barrel syringe may be used to keep two components of a composition until mixing of the two components occurs during administration (e.g., in situ). In some embodiments, the double barrel syringe is attached to a single catheter or needle. In some embodiments, each barrel of the double barrel syringe is attached to a separate needle or catheter. In certain embodiments, the method of treating an infectious disease or ear disease comprises instructing a subject to administer, or providing instructions to a subject for self-administration of, the composition.

A subject for treatment can be any mammal in need of treatment. In certain embodiments, the subject has an ear disease. In some embodiments, the subject has otitis media. In some embodiments, the subject is a human. In certain embodiments, the subject is a domesticated animal, such as a dog, cat, cow, pig, horse, sheep, or goat. In various aspects, the composition is in direct contact with the tympanic membrane for about 1 day to about 30 days. In various aspects, the composition is in contact with the tympanic membrane from about 1 day to about 3 days, from about 3 days to about 7 days, from about 7 days to about 14 days, from about 14 days to about 21 days, or from about 21 days to about 30 days. In various embodiments, the composition forms a sustained release reservoir, in contact with the tympanic membrane. In various aspects, the composition is applied into the ear canal as a liquid, and the composition gels in situ on the surface of the tympanic membrane. When in contact with the tympanic membrane, the therapeutic agent penetrates the tympanic membrane and is delivered to the middle ear. In various embodiments, the delivery across the tympanic membrane is a sustained release of the therapeutic agent over a number of days. The numbers of days that the composition can be in contact with the tympanic membrane can be, but is not limited to, 5 days, 7 days, 10 days, 14 days, 21 days, or 30 days. The number of days that the composition can be in contact with the tympanic membrane can be, but is not limited to, 1-5 days, 1-6 days, 3-6 days, 7-10 days, 7 days, 10 days, 10-15 days, 14 days, 15-20 days, 20-21 days, 15-21 days, 21 days, or 30 days. The composition may be applied singly, or repeatedly in the course of treatment. In various aspects, the composition may be periodically administered from about every 1 day to about every 7 days, from about every 1 day to about every 14 days, or from about every 1 day to about every 30 days. In various embodiments, the composition is naturally extruded from the subject at the end of treatment via natural processes similar to extrusion of ear wax. In certain embodiments, the composition may naturally break down, and its degradation products may be eliminated by the subject. In various embodiments, administration of the compositions described herein comprises adding the matrix forming agent, the permeation enhancer, and the therapeutic agent to the ear canal; then adding a second therapeutic agent to the ear canal; and mixing the matrix forming agent, the permeation enhancer, and the therapeutic agent on the ear canal. In certain embodiments, the second therapeutic agent is an antibiotic agent.

In certain embodiments, a dose is determined based on the minimum inhibitory concentration needed at the site of infection. Without being bound to a particular theory, in various aspects the minimum inhibitory concentration for H. influenza or S. pneumoniae middle ear infections is about 4 μg/mL for ciprofloxacin. In various aspects, a typical dose will require approximately 12 μg of ciprofloxacin, based on an average middle ear volume of 3 mL. In various embodiments, the compositions will comprise sufficient dose to delivery 12 μg of ciprofloxacin to the middle ear.

Without being bound to a particular theory, in various aspects the minimum dosage concentration required for treating pain associated with H. influenza or S. pneumoniae middle ear infections is about 0.36 μg/mL for bupivacaine and/or about 0.32 μg/mL for tetrodotoxin. In various aspects, the minimum dosage concentration achieved (e.g., on the middle ear side during a permeation experiment using dissected ear drum, or in the middle ear) for treating pain associated with H. influenza or S. pneumoniae middle ear infections is about 8 μg/mL (about 25 μM) for bupivacaine and/or about 0.3 ng/mL (about 1 nM) for tetrodotoxin.

In various aspects, the administration of the composition comprises a single application. In other aspects, the administration of the composition comprises multiple applications. For example, the composition may be administered two, three, four, or more times. In certain embodiments, the composition is administered repeatedly until the desired clinical outcome is achieved. For example, the infection is resolved. In certain embodiments, the administration of the composition comprises a first administration of the composition, followed by a second administration of the composition after a period of time. In certain embodiments, the period of time between the first administration of the composition and the second administration of the composition is a week. In certain embodiments, the period of time between the first administration of the composition and the second administration of the composition is more than one week. In certain embodiments, the period of time between the first administration of the composition and the second administration of the composition is one month. In certain embodiments, the period of time between the first administration of the composition and the second administration of the composition is more than one month.

### Kits

Provided herein are kits comprising any of the compositions described herein, which may additionally comprise the compositions in sterile packaging. Provided herein are kits comprising any of the compositions described herein, which may additionally comprise the compositions s in sterile packaging. The kits may comprise two containers for two-part, matrix-forming agents. The therapeutic agent may be included in one or both of the containers of the matrix forming agent, or the therapeutic agent may be packaged separately. The permeation enhancer may be included in one or both of the containers of the matrix forming agent, or the permeation enhancer may be packaged separately. In various aspects the kits may comprise a bottle or bottles, and a dropper or syringe for each bottle.

In certain embodiments, the kit comprises one or more applicators for administering the compositions described herein, for example, droppers (e.g., pipet, eye dropper). In certain embodiments, the kit comprises one or more syringes. In some embodiments, the syringe is pre-loaded with the composition, or one or more component of the composition. In certain embodiments, the kit comprises one or more needle (e.g., blunt-tipped needle). In certain embodiments, the kit comprises one or more catheter (e.g., flexible catheter). In certain embodiments, the kit comprises a double barrel syringe. In some embodiments, the double barrel syringe is pre-loaded with two components of the composition. In some embodiments, the double barrel syringe is attached to a single catheter or needle. In some embodiments, each barrel of the double barrel syringe is attached to a separate needle or catheter.

In certain embodiments, a kit described herein further includes instructions for using the kit, such as instructions for using the kit in a method of the disclosure (e.g., instructions for administering a compound or pharmaceutical composition described herein to a subject). A kit described herein may also include information as required by a regulatory agency such as the U.S. Food and Drug Administration (FDA). In certain embodiments, the kits are used for treating an ear disease condition (e.g., an infectious disease, ear disease, bacterial infection) and/or condition associated with an ear disease comprising one or more containers (e.g., a container) containing a composition described herein, a composition described herein, and instructions for administering the composition to a subject in need thereof. In certain embodiments, the kits further comprise an applicator for administering the compositions described herein, for example, a dropper, syringe, and/or catheter. In certain embodiments, the kits further comprise a dropper, syringe, or catheter. In certain embodiments, the kits comprising formulating an antibiotic (e.g., fluoroquinolone or a beta lactam antibiotic, for example, ciprofloxacin, ceftriaxone) from a powder form within the compositions described herein.

Disclosed herein are exemplary embodiments and combinations thereof:
Embodiment 1. A composition comprising:
   (a) a therapeutic agent or a combination of therapeutic agents;
   (b) a permeation enhancer or a combination of permeation enhancers, wherein the permeation enhancer or combination of permeation enhancers increases the flux of the therapeutic agent or combination of therapeutic agents across a barrier; and
   (c) a block copolymer comprising poloxamer P188;
   wherein:
   the permeation enhancer or combination of permeation enhancers comprises sodium dodecyl sulfate, limonene, and/or bupivacaine, and the sodium dodecyl sulfate comprises between about 0.2% and 3.2% of the composition by weight per volume composition;
   when present, the bupivacaine comprises between about 0.2% and 2.0% of the composition by weight per volume composition;
   the limonene comprises between about 0.5% and 7.0% of the composition by weight per volume composition; and
   the poloxamer P188 comprises between about 18% and about 62% of the composition by weight per volume composition;
   wherein:
   the composition forms a gel at temperatures above a phase transition temperature; and
   the phase transition temperature is less than about 37° C.
Embodiment 2. The composition of embodiment 1, wherein:
   (c) the block copolymer is poloxamer P188;
   the permeation enhancer or combination of permeation enhancers comprises sodium dodecyl sulfate, limonene, and/or bupivacaine, and
   the sodium dodecyl sulfate comprises between about 0.5% and 3.2% of the composition by weight per volume composition;
   the bupivacaine comprises between about 0.25% and 0.75% of the composition by weight per volume composition;
   the limonene comprises between about 1.5% and 6.0% of the composition by weight per volume composition; and
   the poloxamer P188 comprises between about 20% and about 50% of the composition by weight per volume composition;
   wherein:
   the composition forms a gel at temperatures above a phase transition temperature; and
   the phase transition temperature is between about 20° C. and about 37° C.
Embodiment 3. The composition of embodiment 1 or 2, wherein the composition comprises the permeation enhancer limonene.
Embodiment 4. The composition of any one of embodiments 1-3, wherein the composition comprises the permeation enhancers sodium dodecyl sulfate and limonene.
Embodiment 5. The composition of any one of embodiments 1, 3, or 4, wherein the permeation enhancers consist of sodium dodecyl sulfate and limonene.
Embodiment 6. The composition of any one of embodiments 1-4, wherein the composition comprises the permeation enhancers sodium dodecyl sulfate, limonene, and bupivacaine.
Embodiment 7. The composition of any one of embodiments 1-6, wherein the composition comprises between about 1.5-3.2% wt/vol of sodium dodecyl sulfate.
Embodiment 8. The composition of any one of embodiments 1-7, wherein the composition comprises between about 0.2-1.0% wt/vol of sodium dodecyl sulfate.
Embodiment 9. The composition of any one of embodiments 1-7, wherein the composition comprises between about 1.0-2.7% wt/vol of sodium dodecyl sulfate.
Embodiment 10. The composition of any one of embodiments 1-7 or 9, wherein the composition comprises between about 1.8-2.7% wt/vol of sodium dodecyl sulfate.
Embodiment 11. The composition of any one of embodiments 1-7, 9, or 10, wherein the composition comprises between about 2.0-2.6% wt/vol of sodium dodecyl sulfate.
Embodiment 12. The composition of any one of embodiments 1-7, wherein the composition comprises between about 0.5-1.5% wt/vol of sodium dodecyl sulfate.
Embodiment 13. The composition of any one of embodiments 1-9 or 12, wherein the composition comprises about 1.0% wt/vol of sodium dodecyl sulfate.
Embodiment 14. The composition of any one of embodiments 1, 3, 4, or 6-13, wherein the composition comprises between about 0.5-1.25% wt/vol of bupivacaine.
Embodiment 15. The composition of any one of embodiments 1-4 or 6-13, wherein the composition comprises between about 0.3-0.6% wt/vol of bupivacaine.
Embodiment 16. The composition of any one of embodiments 1, 3, 4, or 6-13, wherein the composition comprises about 1.0-1.25% wt/vol of bupivacaine.
Embodiment 17. The composition of any one of embodiments 1-4 or 6-15, wherein the composition comprises about 0.5% wt/vol of bupivacaine.
Embodiment 18. The composition of embodiment 1, 3, 4, 6-14, or 16, wherein the composition comprises about 1.0% wt/vol of bupivacaine.
Embodiment 19. The composition of any one of embodiments 1-18, wherein the composition comprises between about 4.0-6.0% wt/vol of limonene.
Embodiment 20. The composition of any one of embodiments 1-19, wherein the composition comprises between about 4.0-5.0% wt/vol of limonene.
Embodiment 21. The composition of any one of embodiments 1-18, wherein the composition comprises about 0.5-3.5% wt/vol of limonene.
Embodiment 22. The composition of any one of embodiments 1-17 or 21, wherein the composition comprises about 2.0% wt/vol of limonene.
Embodiment 23. The composition of any one of embodiments 1-20, wherein the composition comprises about 4.0% wt/vol of limonene.
Embodiment 24. The composition of any one of embodiments 1-20, wherein the composition comprises about 5.0% wt/vol of limonene.
Embodiment 25. The composition of any one of embodiments 1-24, wherein the composition comprises between about 21.0-37.0% wt/vol of poloxamer P188.
Embodiment 26. The composition of any one of embodiments 1-24, wherein the composition comprises between about 30.0-50.0% wt/vol of poloxamer P188.
Embodiment 27. The composition of any one of embodiments 1-26, wherein the composition comprises between about 34.0-37.0% wt/vol of poloxamer P188.
Embodiment 28. The composition of any one of embodiments 1-24 or 26, wherein the composition comprises between about 40.0-50.0% wt/vol of poloxamer P188.
Embodiment 29. The composition of any one of embodiments 1-27, wherein the composition comprises about 21.0% wt/vol, about 23.0% wt/vol, about 24.0% wt/vol, about 25.0% wt/vol, about 26.0% wt/vol, or about 35.0% wt/vol of poloxamer P188.
Embodiment 30. The composition of any one of embodiments 1-26 or 28, wherein the composition comprises between about 45.0% wt/vol of poloxamer P188.
Embodiment 31. The composition of any one of embodiments 1-30, wherein the phase transition temperature is between about 24.0° C. and about 37.0° C.
Embodiment 32. The composition of any one of embodiments 1-31, wherein the phase transition temperature is between about 29.0° C. and about 36.5° C.
Embodiment 33. The composition of any one of embodiments 1-30, wherein the phase transition temperature is between about 20.0° C. and about 30.0° C.
Embodiment 34. The composition of any one of embodiments 1-30 or 33, wherein the phase transition temperature is about 25.0° C.
Embodiment 35. The composition of any one of embodiments 1-34, wherein the phase transition temperature is about 25.0° C., about 27.0° C., about 30.0° C., about 31.0° C., about 33.0° C., about 34.0° C., or about 35.5° C.
Embodiment 36. The composition of any one of embodiments 1-35, wherein the therapeutic agent is an antibiotic agent, anesthetic agent, anti-inflammatory agent, analgesic agent, anti-fibrotic agent, anti-sclerotic agent, anticoagulant agent, or diagnostic agent.
Embodiment 37. The composition of embodiment 36, wherein the antibiotic agent is a fluoroquinolone or a beta lactam antibiotic.
Embodiment 38. The composition of embodiment 36 or 37, wherein the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole.
Embodiment 39. The composition of any one of embodiments 36-38, wherein the antibiotic agent is ciprofloxacin or ceftriaxone.
Embodiment 40. The composition of any one of embodiments 36-39, wherein the composition comprises between about 3.0-50.0% wt/vol of the antibiotic agent that is ciprofloxacin or ceftriaxone.
Embodiment 41. The composition of any one of embodiments 36-39, wherein the antibiotic agent is ciprofloxacin.
Embodiment 42. The composition of embodiment 41, wherein the composition comprises between about 1.0-5.0% wt/vol of ciprofloxacin.
Embodiment 43. The composition of any one of embodiments 36-38, wherein the antibiotic is ciprofloxacin, gatifloxacin, ceftriaxone, gemifloxacin, moxalactam, levofloxacin, meropenem, or ampicillin.
Embodiment 44. The composition of any one of embodiments 1-43, wherein the composition comprises:
   about 1.5-3.2% wt/vol of sodium dodecyl sulfate;
   about 2.0-5.0% wt/vol of limonene;
   about 21.0-37.0% wt/vol of poloxamer P188; and
   about 4.0-45.0% wt/vol of antibiotic.
Embodiment 45. The composition of any one of embodiments 1-44, wherein the composition comprises:
   about 2.0-3.1% wt/vol of sodium dodecyl sulfate;
   about 4.0-5.0% wt/vol of limonene;
   about 21.0-36.0% wt/vol of poloxamer P188; and
   about 16.0-43.0% wt/vol of antibiotic.
Embodiment 46. The composition of any one of embodiments 1-44, wherein the composition comprises:
   about 2.0-3.1% wt/vol of sodium dodecyl sulfate;
   about 4.0-5.0% wt/vol of limonene;
   about 21.0-36.0% wt/vol of poloxamer P188; and
   about 16.0-43.0% wt/vol of ceftriaxone.
Embodiment 47. The composition of any one of embodiments 1-43, wherein the composition comprises:
   about 1.5-3.2% wt/vol of sodium dodecyl sulfate;
   about 3.0-5.0% wt/vol of limonene;
   about 34.0-37.0% wt/vol of poloxamer P188; and
   about 2.0-5.0% wt/vol of antibiotic.
Embodiment 48. The composition of any one of embodiments 1-43 or 47, wherein the composition comprises:
   about 1.0% wt/vol of sodium dodecyl sulfate;
   about 0.5% wt/vol of bupivacaine;
   about 2.0% wt/vol of limonene; and
   about 45.0% wt/vol of poloxamer P188.
Embodiment 49. The composition of any one of embodiments 1-43, 47, or 48, wherein the composition comprises:
   about 1.0% wt/vol of sodium dodecyl sulfate;
   about 0.5% wt/vol of bupivacaine;
   about 2.0% wt/vol of limonene;
   about 45.0% wt/vol of poloxamer P188; and
   about 2.0-5.0% wt/vol of ciprofloxacin.
Embodiment 50. A pharmaceutical composition comprising a composition of any one of embodiments 1-49, and optionally a pharmaceutically acceptable excipient.
Embodiment 51. The pharmaceutical composition of embodiment 50, wherein the pharmaceutical composition comprises a therapeutically effective amount of the composition for use in treating a disease or condition in a subject in need thereof.
Embodiment 52. A method of treating a disease or condition in a subject in need thereof, the method comprising administering to the subject a therapeutically effective amount of a composition of any one of embodiments 1-49, or pharmaceutical composition of embodiment 50 or 51.
Embodiment 53. The pharmaceutical composition of embodiment 51, wherein the disease is an infectious disease.
Embodiment 54. The pharmaceutical composition of embodiment 53, wherein the infectious disease is a bacterial infection.
Embodiment 55. The pharmaceutical composition of embodiment 54, wherein the bacterial infection is an H. influenzae, S. pneumoniae, or M. catarrhalis infection.
Embodiment 56. The pharmaceutical composition of embodiment 51, wherein the disease is an ear disease.
Embodiment 57. The pharmaceutical composition of embodiment 53, wherein the infectious disease is otitis media.
Embodiment 58. The method of embodiment 52, wherein the disease is an infectious disease.
Embodiment 59. The method of embodiment 58, wherein the infectious disease is a bacterial infection.
Embodiment 60. The method of embodiment 59, wherein the bacterial infection is an H. influenzae, S. pneumoniae, or M. catarrhalis infection.
Embodiment 61. The method of embodiment 52, wherein the disease is an ear disease.
Embodiment 62. The method of embodiment 58, wherein the infectious disease is otitis media.
Embodiment 63. A method of delivering a composition of any one of embodiments 1-49, the method comprising administering the composition to an ear canal of a subject.
Embodiment 64. The method of embodiment 63, wherein the composition contacts the surface of a tympanic membrane.
Embodiment 65. The method of embodiment 64, wherein the administering comprises placing drops of the composition into the ear canal, or placing a dose of the composition into the ear canal using a catheter.
Embodiment 66. The method of any one of embodiments 63-65, wherein the administering comprises using an applicator to place the composition into the ear canal.
Embodiment 67. The method of any one of embodiments 63-66, wherein the administering comprises placing the composition into the ear canal with a syringe.
Embodiment 68. Use of a composition to treat and/or prevent a disease or condition in a subject in need thereof, the use comprising administering to the subject a therapeutically effective amount of a composition of any one of embodiments 1-49, or pharmaceutical composition of embodiment 50 or 51.
Embodiment 69. A kit for treating an ear disease and/or condition associated with an ear disease comprising a container, a composition of any one of embodiments 1-49, and instructions for administering the composition to a subject in need thereof.
Embodiment 70. The kit of embodiment 69, further comprising a dropper, syringe, or catheter.
Embodiment 71. The kit of embodiment 69 or 70, comprising formulating an antibiotic from a powder form in the composition of any one of embodiments 1-49.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1

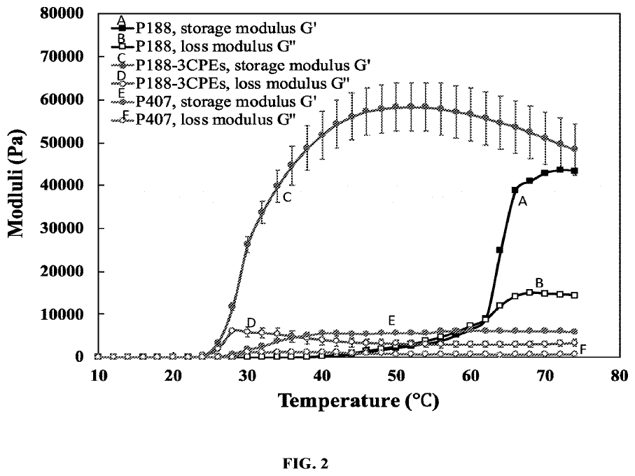

Herein is a composition comprising a FDA-approved polymer (P188) that does not gel at temperatures in the physiological range, where this composition is made to gel at below body temperature. A hydrogel based drug delivery system containing the model antibiotic ciprofloxacin (Cip), “3CPEs,” and copolymer poloxamer P188 was designed. Specifically, the “3CPEs” refer to 2% (w/v) limonene (LIM), 1% (w/v) sodium dodecyl sulfate (SDS) and 0.5% (w/v) bupivacaine (BUP), which is the exemplary optimized CPEs formulation with high permeation enhancement effect and low tissue irritation 1-2. The viscoelastic studies showed that the storage modulus (G′) became higher than loss modulus (G″), an indication of solution to gel transition (sol-gel), at around 63° C. for 45% [P188] (FIG. 2). The preliminary studies also found that the sol-gel transition temperature of P188 kept constant (higher than 60° C.) regardless of changing the poloxamer concentration. Surprisingly, the introduction of 3CPEs to 45% [P188] sharply decrease the gelation temperature as low as to 24° C., which is slightly higher than room temperature and lower than body temperature. The gelation properties of a formulation with P407, a widely used thermoreversible poloxamer for local drug delivery, were also compared. The storage modulus (G′) of 3CPEs-45%[P188] (˜45 k Pa) is much higher than 18%[P407] (˜8 k) at body temperature (FIG. 2). High mechanical strength of gel matrix is valuable in the application of local drug delivery. The hydrogel formulation is expected to flow into place and then promptly become a gel at body temperature with maintaining physical integrity without dropping out throughout the course of treatment.

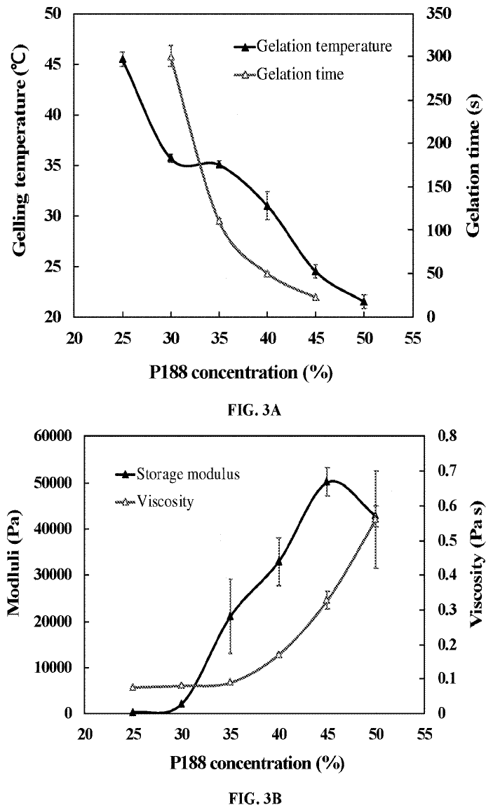

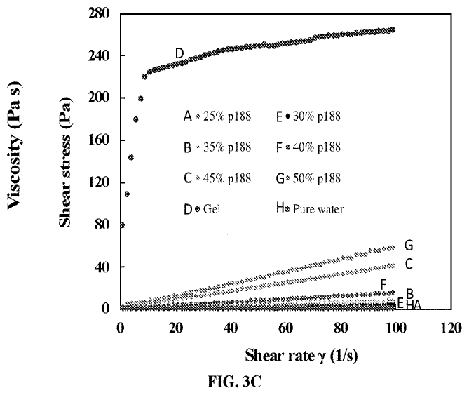

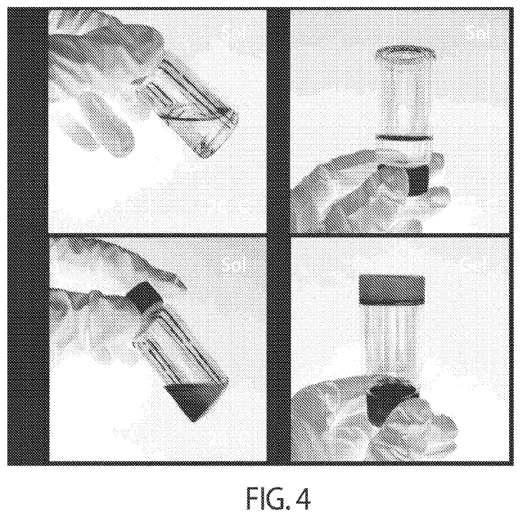

The effect of poloxamer concentration on the rheological properties of formulation is shown in FIGS. 3A-3C. It was found that the increase of poloxamer concentration resulted in the decrease of gelation time and gelling temperature (FIG. 3A), and the increase of viscosity at room temperature and storage modulus at body temperature of the formulation (FIG. 3B). The viscosity of [P188]-3CPEs at room temperature was slightly higher than pure water but much low than poloxamer gel (i.e., P407) at 37° C. (FIG. 3C). A formulation of 45% [P188]-3CPEs was selected in the study because the sol-gel transition temperature of this formulation is around 24.5° C., which is higher than room temperature but lower than body temperature. The formulation of 45% [P188]-3CPEs can freely flow in the tube at room temperature, and gel quickly (˜22 s) at body temperature (FIG. 4).

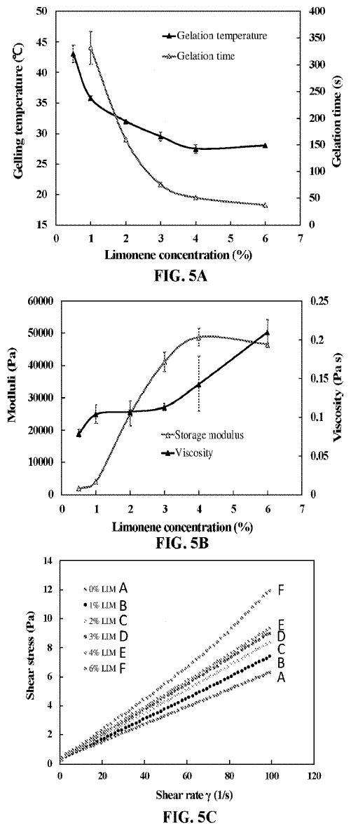

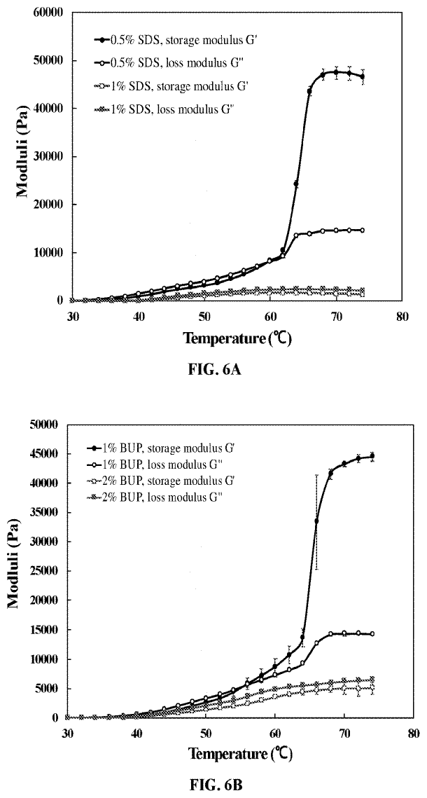

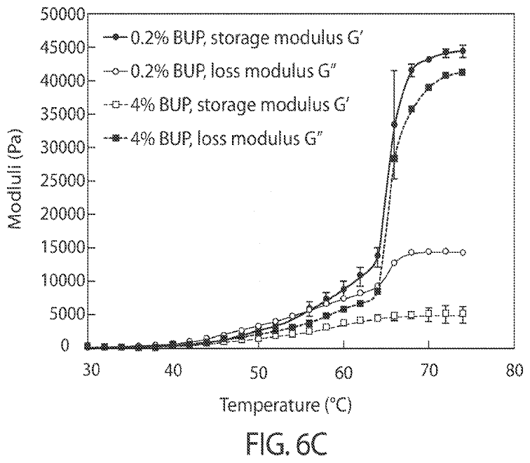

Different concentrations of CPE components were then separately added into the formulation of 45% [P188] to evaluate the effect of individual CPEs on the rheological properties of formulation. As shown in FIGS. 5A-5B, the increase of limonene concentration resulted in the decrease of gelation time and gelling temperature (FIG. 5A), and the increase of viscosity at room temperature and storage modulus at body temperature of the formulation (FIG. 5B). The addition of either SDS or BUP at high concentration (i.e., 1%) greatly decreased the G′ and G″ of the formulation. Meanwhile, G′ changed to be lower than G″ over the temperature range of 30° C. to 75° C. (FIGS. 6A-6B), which suggested that both SDS and BUP could inhibit the reverse thermal gelation properties of P188.

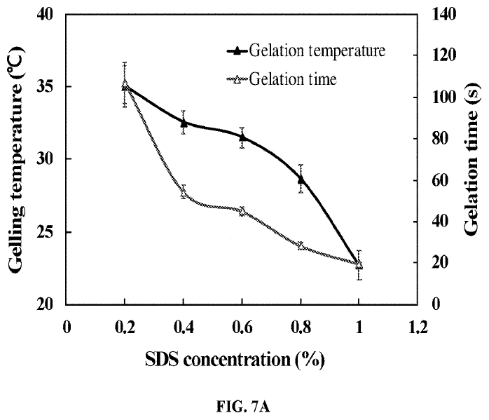

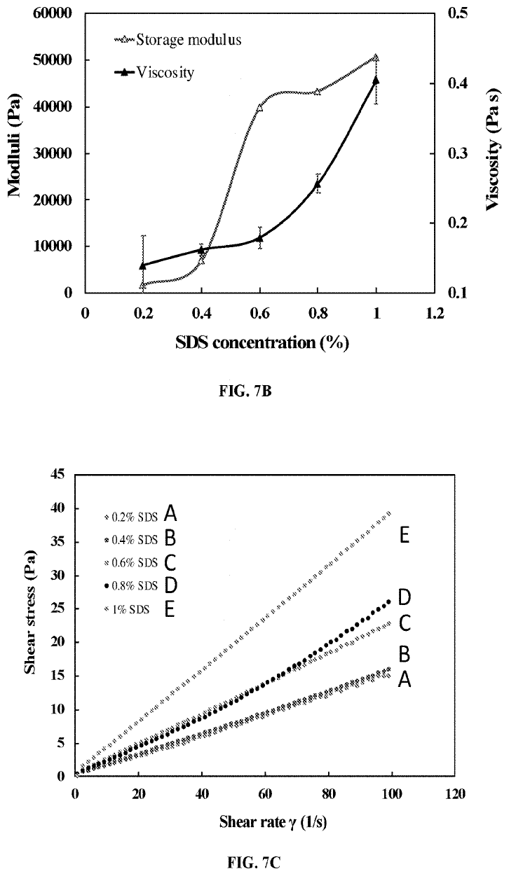

Interestingly, the addition of SDS in 2%-LIM-45% [P188] showed a similar effect as LIM, that is decreasing gelation time and gelling temperature, and increasing the formulation viscosity at room temperature and storage modulus at body temperature (FIGS. 7A-7C). The BUP showed the opposite effect after the addition in 2%-LIM-45% [P188]. Overall, the results indicated that the influences of CPEs were dominated by the effects of LIM, and adjusted by SDS and BUP.

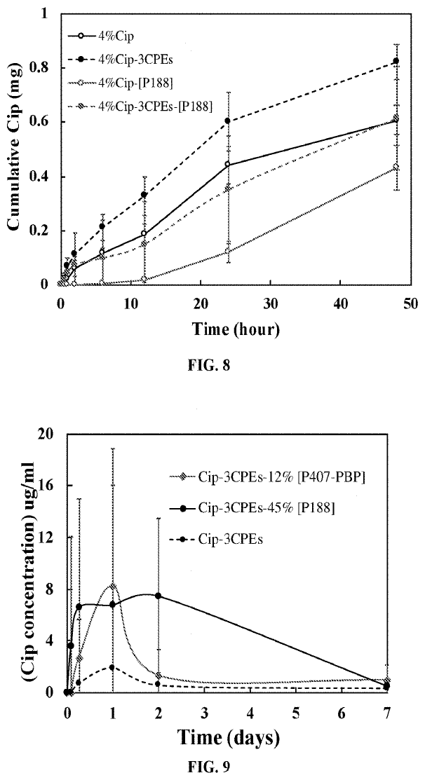

The drug transport was evaluated across the TM ex vivo in auditory bullae excised from healthy chinchillas. The presence of 3CPEs enhanced the flux of 4% Cip across the TM during the course of permeation experiment. Conversely, the inclusion of a hydrogel (i.e., P188) tended to decrease the drug flux across the TM. However, this retardation effect could be overcome by the addition of CPEs (FIG. 8).

The drug transport across the TM was then evaluated using an in vivo model. OM was induced by direct inoculation of NTHi into the middle ear of chinchillas. The ciprofloxacin concentration was determined in the middle ear fluid during the course of treatment. The concentration of ciprofloxacin peaked after 2 days for the animals treated with Cip-3CPEs-45% [P188] (7.48 μg/mL) and 1 day for Cip-3CPEs-[P407-PBP] treatment (8.23 μg/mL) (FIG. 9). However, the ciprofloxacin concentration was higher for the animals treated with Cip-3CPEs-45% [P188] than Cip-3CPEs-[P407-PBP] at 2 hours, 6 hours, 1 day and 2 days after hydrogel administration. After 7 days' treatment, the ciprofloxacin concentration was 0.602 and 0.927 μg/mL in the middle ear fluid of animals treated with Cip-3CPE-45% [P188] and Cip-3CPE-[P407-PBP], respectively, which are still higher than minimum inhibitory concentration (MIC) of ciprofloxacin for treating NTHi (0.1 to 0.5 mg/ml)³.

The therapeutic effect of the formulations on NTHi induced otitis media (OM) was then studied in chinchillas. The formulation with [P407-PBP]

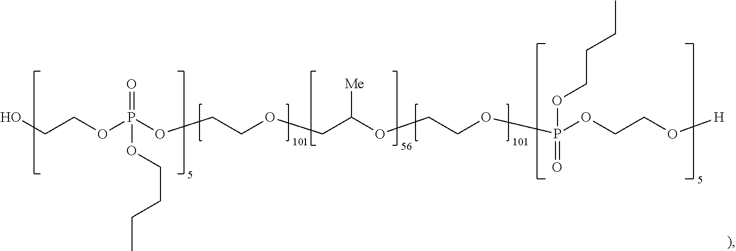

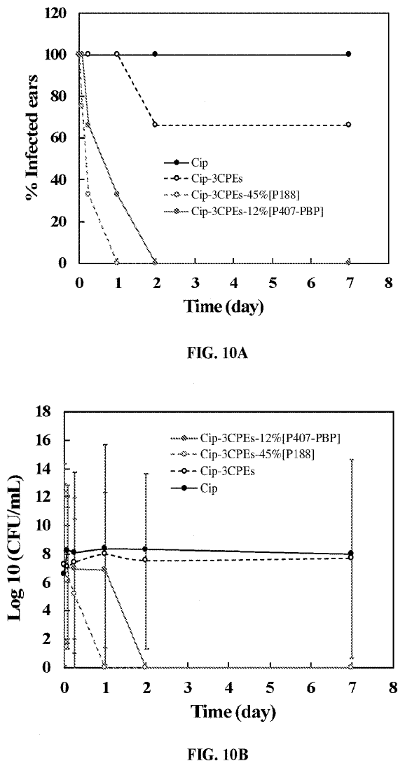

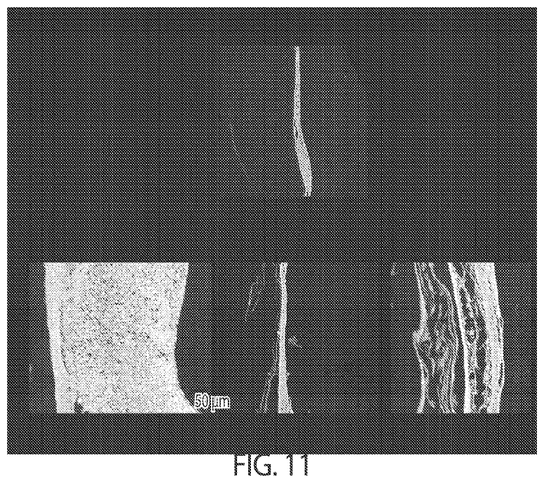

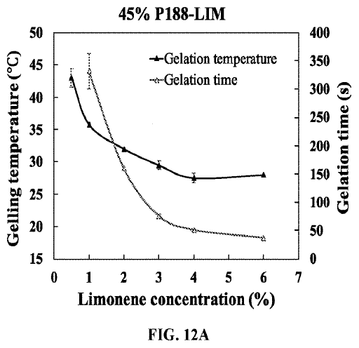

a previously developed chemical modified poloxamer, that gels with CPEs, was also compared with the new formulation. The cure rate was lower for the animals treated with 4% Cip and 4% Cip-3CPE, infection was detectable in 100% animals and 66.7% animals by day 7 (FIG. 10A). The average number of colonies (CFU/ml) in middle ear remained consistently high during the time course of 7 days for both treatment. The amount of colonies was reduced on day 1 and no colonies were detected on day 2 after the application of the formulation of Cip-3CPE-12%[P407-PBP] (FIG. 10B). These results are in agreement with the finding from a previous study.⁴ The formulation of Cip-3CPE-P188 showed even better therapeutic effect, in that no colonies were detected on day 1 after the application of this formulation.

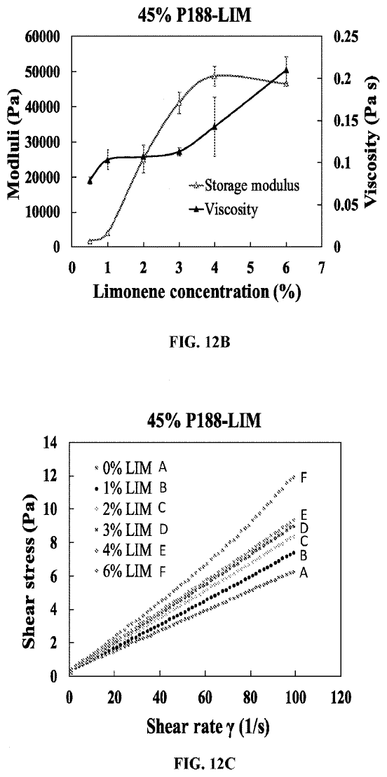

H&E (hematoxylin and eosin)-stained sections of infected TMs treated with the gel formulation looked very similar with the TMs from healthy animals, indicating the TM does return to normal status. Conversely, the infected TMs were much thicker than healthy TM without gel treatment (i.e., untreated and Cip treatment), which was due to inflammation from the infection (FIG. 11).

The results also suggested that no Ciprofloxacin was detectable in the bloodstream at any time point throughout the course of treatment, which provided strong evidence for low or no systemic exposure of antibiotics (see Table. 1).

Overall, the composition described in this example may be used in the application of effective sustained local therapy for OM to address issues relating to subject compliance in using administered therapeutic agents, for example, antibiotics, and minimization of systemic exposure to such therapeutic agents.

TABLE 1
Systemic exposure to ciprofloxacin. Blood samples were analyzed
for ciprofloxacin content by HPLC at predetermined intervals
after gel application (i.e., 4% Cip-3CPEs-45% [P188]).

| | Concentration of |
|---|---|
| | ciprofloxacin in |
| Time | plasma (μg) |
| 0 | Not detected |
| 2 hours | Not detected |
| 6 hours | Not detected |
| 1 day | Not detected |
| 2 days | Not detected |
| 7 days | Not detected |

### Example 2

The storage and loss moduli of the formulations were measured every 2° C. during a temperature sweep from 10° C. to 70° C. The temperature at which the storage modulus (G′) exceeds the loss modulus (G″) is considered the gelation temperature. To measure gelation time, formulations in scintillation vials were immersed in a 37° C. water bath over a stir plate. The time until the stir bar stops rotating was noted as the gelation time. Different concentration of CPEs (individual or combination) were added in 45% P188 to evaluate the effect of CPEs on the temperature-dependent mechanical properties of P188 gel.

The increase of limonene concentration within 45% P188 resulted in the decrease of gelation temperature and gelation time (see FIG. 12A), and the increase of viscosity at room temperature and storage modulus at body temperature of the formulations (see FIG. 12B). For example, a formulation of 45% P188-1% Lim formed a gel at around 35.7° C. and the gelation time at body temperature was 332 seconds (s); a formulation of 45% P188-6% Lim gelled at around 28° C. and the gelation time at body temperature was 37 s. The viscosity of a formulation of 45% P188-1% Lim was around 80 mPas (similar with concentrated milk) and the viscosity of a formulation of 45% P188-6% Lim was 210 mPas (similar with latex emulsion).

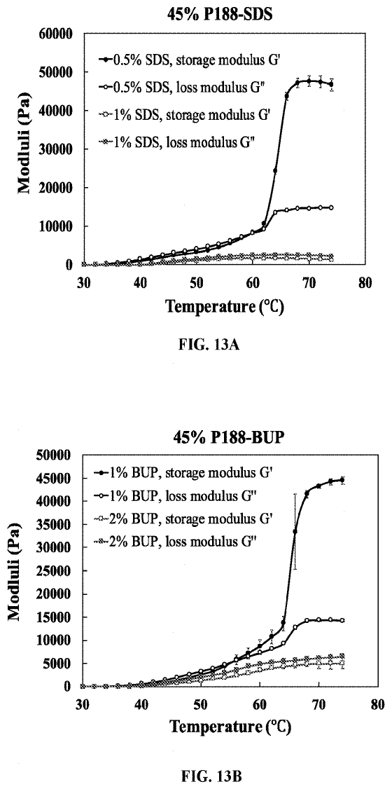

Adding a high concentration of single SDS or bupivacaine suppressed the gelation of 45% P188 formulation, e.g., the loss modulus (G″) was higher than the storage modulus (G′) when 1% SDS or 2% bupivacaine were added to the P188 solution; that is, the material did not form a gel in the presence of high concentration of SDS or bupivacaine (see FIGS. 13A-13B).

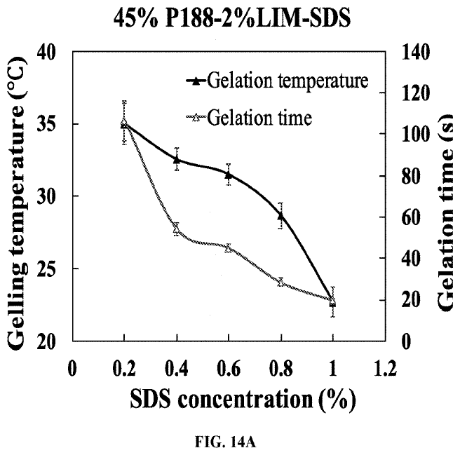

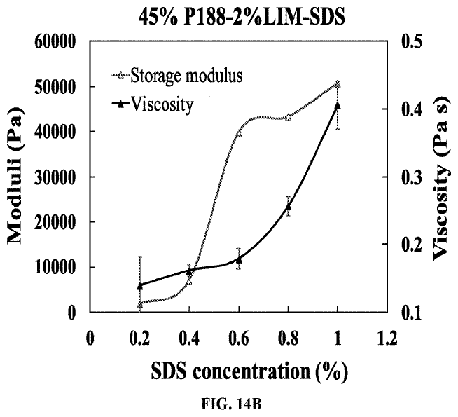

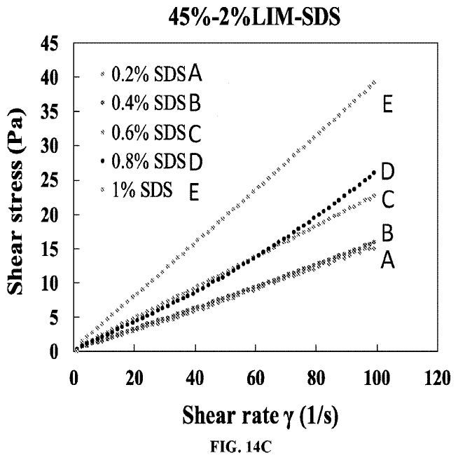

In the presence of limonene, the increase of SDS concentration within the 45% P188-2% Lim formulation resulted in the decrease of gelation temperature and gelation time (see FIG. 14A), and the increase of viscosity at room temperature and storage modulus at body temperature of the formulations (FIG. 14B). For example, a formulation of 45% P188-2% Lim-0.2% SDS formed a gel at around 35° C. and the gelation time at body temperature was 107 s; 45% P188-2% Lim-1% SDS gelled at around 22.7° C. and the gelation time at body temperature was 19 s. The viscosity of the formulation of 45% P188-2% Lim-0.2% SDS was around 140 mPas (similar with liquid egg) and the viscosity of the formulation of 45% P188-2% Lim-1% SDS was 400 mPas (similar with pottage).

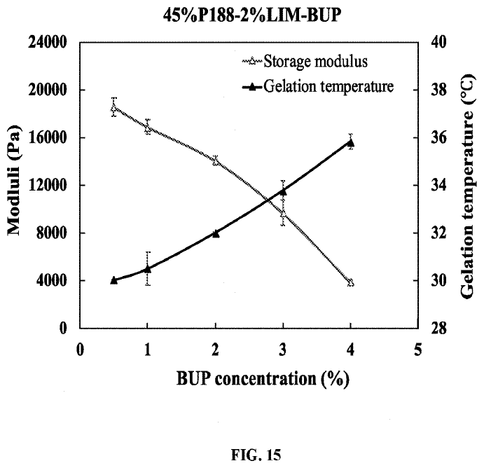

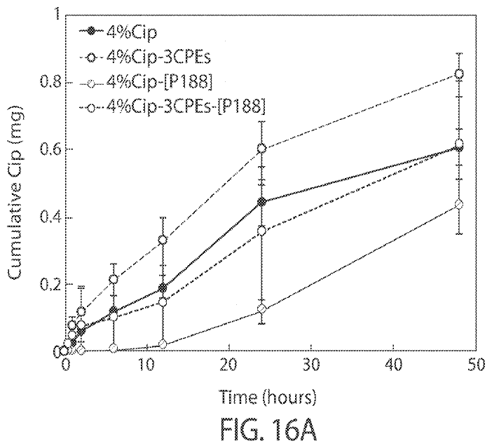

In the presence of limonene, it appears that the increase of bupivacaine concentration within the formulation of 45% P188-2% Lim resulted in the increase of gelation temperature (also gelation time, data not shown) and the decrease of storage modulus at body temperature of the formulations (45% P188-2% Lim-Bup) (FIG. 15). The formulation did not form a gel in the presence of high concentration of bupivacaine.

### Example 3

Poloxamers are nonionic triblock copolymers composed of a central hydrophobic chain (poly(propylene glycol)) or PPG flanked by two hydrophilic chains of (poly(ethylene glycol)) or PEG. All of the poloxamers share the same structural scaffold (shown below), but have different numbers of x, y and z.

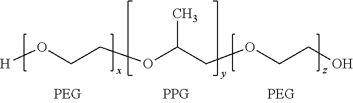

Eight different poloxamers (shown below in List 1B) were tested with CPE agents (i.e., SDS, bupivacaine and limonene), and/or with the exemplary CPE(s) as outlined below using the conditions shown in List 1A. Specifically, the dose-response curves were prepared and analyzed for the addition of exemplary single CPEs or the combination of CPEs outlined below on the gelation properties of formulations (compositions) with the different poloxamers. None of these exemplary formulations with the eight exemplary tested poloxamers showed thermal-reversible gelation properties, where the formulation would stay as a solution at lower temperature, but gels at elevated temperature. The composition and concentrations of the CPEs tested are listed as follows:

### Exemplary Formulations and Concentrations of CPEs (List 1A):

1. Single addition of limonene from 0.5% to 20%.
2. Single addition of SDS from 0.2% to 10%.
3. Addition of fixed limonene concentration (2%), but different SDS concentration from 0.2% to 10%.
4. Addition of fixed limonene concentration (2%), but different bupivacaine concentration from 0.5% to 5%.
5. Addition of fixed limonene (2%) and SDS (1%) concentration, but different bupivacaine concentration from 0.5% to 5%.

### The Eight Poloxamers Tested in the Exemplary Formulations (List 1B) Include:

1. P182 (Pluronic L62). Properties: x=8, y=30, z=8;
2. P331 (Pluronic L101). Properties: x=7, y=54, z=7
3. P124 (Pluronic L44). Properties: x=10, y=23, z=10
4. P401 (Pluronic L121). Properties: x=5, y=68, z=5
5. P181 (Pluronic L-61). x and z=2; y=31
6. P231 (Pluronic L-81). x and z=3; y=43
7. P338 (Pluronic F-108). x and z=141; y=44
8. Pluronic 31R1. x and z=2; y=31

Formulations using the conditions of List 1A, with each of the above-noted poloxamers were studied using different concentrations varying from 10% to 50% of the poloxamers in List 1B. With regard to the concentration ranges of the poloxamers in the formulations, the compositions and concentrations of the CPEs might be adjusted to control the poloxamer (e.g., P188) concentration in the formulation.

### Example 3A

### The Eight Poloxamers Tested in the Exemplary Formulations for Example 3A Include:

1. P182 (Pluronic L62). Properties: x=8, y=30, z=8;
2. P331 (Pluronic L101). Properties: x=7, y=54, z=7
3. P124 (Pluronic L44). Properties: x=10, y=23, z=10
4. P401 (Pluronic L121). Properties: x=5, y=68, z=5
5. P181 (Pluronic L-61). x and z=2; y=31
6. P231 (Pluronic L-81). x and z=3; y=43
7. Pluronic 31R1. x and z=2; y=31
8. Pluronic L31.

For these poloxamers, two concentrations of poloxamers were studied including 20% and 45%.

### Exemplary Formulations and Concentrations of CPEs (for Example 3A)

1. Single addition of limonene from 0.5% to 10%.
2. Single addition of SDS from 0.2% to 10%.
3. Addition of fixed limonene concentration (2%), but different SDS concentration from 0.2% to 10%.

### Results

The P331, P401, P231 and P181 formulations were insoluble after single addition of limonene.

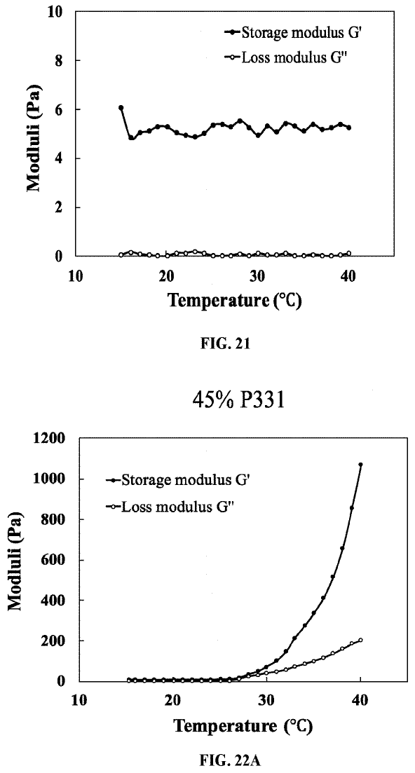

For all other tested formulations (except P331), the rheology as a function of temperature is shown in FIG. 21. All these formulations (different poloxamers with addition of different concentrations of CPEs) showed the similar rheologic properties, that both storage modulus (G′) and loss modulus (G″) were less than 10 Pa over the temperature range of 15° to 40° C., indicating no gel were formed.

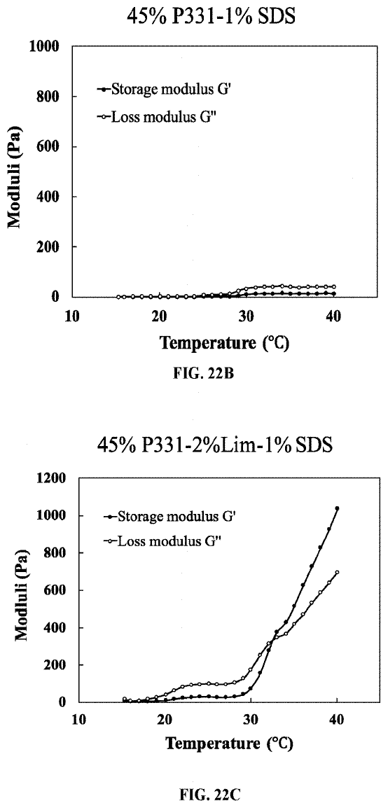

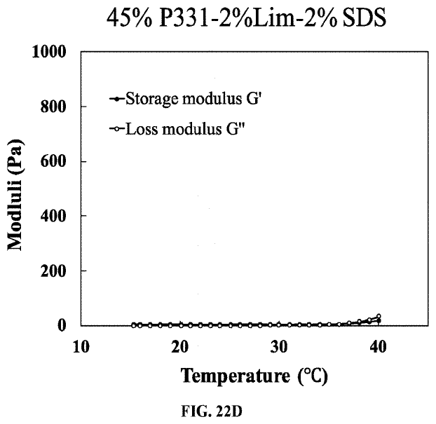

For P331 formulations, the rheology as a function of temperature is shown in FIGS. 22A-22D. Both storage modulus (G′) and loss modulus (G″) of P331 were less than 1.2 kPa over the temperature range of 15° to 40° C., indicating no gel were formed (G′ has to be higher than 2 kPa for the gel formation) (FIG. 22A). The single addition of SDS could suppress the gelation properties of P331 formulation: the loss modulus (G″) was higher than storage modulus (G′) after addition of 1% SDS (FIG. 22B). In presence of 2% Lim, the addition of SDS at low concentration (<1%) did not significantly change the storage modulus (G′) of formulations (FIG. 22C). Further increasing the SDS concentration to 2% could suppress the gelation properties of P331 formulation (FIG. 22D).

Overall, none of these formulations showed thermal-reversible gelation properties, where the formulation would stay as a solution at lower temperature, but gels at elevated temperature.

### Example 4

Exemplary formulations (e.g., formulations 1-6) with the specified concentrations of poloxamer (poloxamer P188), sodium dodecyl sulfate (SDS), and limonene (LIM), that also include a therapeutic agent are shown in Table 2 below. G′ indicates the storage modulus, and G″ indicates the loss modulus.

TABLE 2
Exemplary formulations

| | | | | Gelation | | | | | Gelation |
|---|---|---|---|---|---|---|---|---|---|
| Formulation | P188 | SDS | LIM | temp | | G′ at | | Viscosity | time at |
| No. | concentration | concentration | concentration | (G′ > G″) | | 37° C. | | (Pa*s) | 37° C. |
| 1 | 35% | 1.8% | 5% | 34.02° | C. | 4687 | Pa | 0.201 | 32 s |
| 2 | 35% | 2% | 5% | 35.1° | C. | 4705.9 | Pa | 0.198 | 35 s |
| 3 | 35% | 2.8% | 4% | 35.5° | C. | 7718.2 | Pa | 0.35 | 31 s |
| 4 | 35% | 3% | 4% | 34° | C. | 8294.8 | Pa | 0.336 | 28 s |
| 5 | 37% | 2.6% | 5% | 29° | C. | 44788 | Pa | 0.62 | 12 s |
| 6 | 37% | 2.8% | 5% | 36.5° | C. | 12256.4 | Pa | 0.48 | 1 min 2 s |
| 7 | 37% | 3% | 5% | 33.5° | C. | 33602.4 | Pa | 0.581 | 30 s |

### Example 5

Thermoreversible polymers that undergo sol-gel transition in a physiological temperature range have been used in biomedical applications. Here, a thermoreversible platform for drug delivery across the biological barriers was developed by a combination of the chemical permeation enhancers (CPEs) with poloxamer 188. The hydrogels formed by P188-CPEs showed stronger mechanical strength than P407. Two CPEs including limonene (LIM) and sodium dodecyl sulfate (SDS) were useful to make the P188 gel at temperatures in the physiological range. Both SDS and LIM may promote the P188 micelle formation through an entropy driven process. SDS may bind to the P188 and break down the micelles into small micellar aggregates with higher number density, and LIM may load into the core of micelle and compensate the reduced sphere radius of P188-SDS aggregates, which promoted the gel formation and decreased the initial gelation temperature by increasing overall volume fraction of P188-SDS aggregates. This thermoreversible gel formulation enabled the flux of antibiotic ciprofloxacin across the tympanic membrane (TM) and completely eradicated otitis media from nontypable Haemophilus influenzae (NTHi) in chinchillas after single administration. This system could be applicable in a broad range of applications of sustained drug delivery, especially drug delivery across biological barriers.

### Introduction

Materials with reverse thermal gelation (thermoreversible gels) are used in drug delivery. As solutions at room temperature, they are easy to apply even through a small gauge applicator, while at a higher temperature (typically between 25 and 37° C.), gelation occurs, increases the durations of drug releaser and tissue retention. Exemplary materials are poloxamers, i.e., ABA block copolymers where A=poly(ethylene oxide) and B=poly(propylene oxide).

The challenge is to improve the physical properties of the hydrogel without expensive and time-consuming preparation procedures, ¹² and without compromising the desirable mechanical and drug-delivery properties.

The importance of those properties is highlighted in the context of non-invasive trans-tympanic drug delivery. Formulations in which drugs were able to cross the tympanic membrane (TM) from a thermoreversible gel, due to the presence of CPEs have been previously demonstrated. A single dose of this therapeutic platform delivering the antibiotic ciprofloxacin could eradicate otitis media (OM) in a chinchilla model after administration of a single dose.¹³ There, the obvious challenge is in getting drugs across a barrier—the tympanic membrane (TM)—that has very low permeability to most molecules. However, additional design constraints are created by the nature of the target patient population—most of whom are less than two years old and are therefore unlikely to stay still for extended periods—and by the need to provide therapy for approximately one week. Consequently, it would be better if the drug delivery system was a low viscosity liquid at room temperature so that it could be applied easily and flow down the external auditory canal to the TM. It has to gel rapidly upon contact with the warm tympanic membrane and have mechanical properties strong enough that it remains at the TM to provide treatment over several days. Moreover, the gel cannot be so viscous that it slows efflux of the drug to the point that flux across the TM is inadequate.

Here, the fact that CPEs can alter the rheological properties of poloxamers and their derivatives¹³ was built upon to develop a thermoresponsive poloxamer-based gel with strong mechanical properties in the presence of CPEs, while maintaining low viscosity at room temperature and rapid gelation, and providing therapeutically effective flux of ciprofloxacin across the TM. The effects of the CPEs on the ultrastructure of the gel were elucidated to explain their effects on the mechanical properties.

### Results and Discussion

### Rheological Properties

To identify poloxamers whose mechanical properties might be enhanced by CPEs, a screen was conducted of the rheological properties of a range of poloxamers, with and without the addition of a combination (termed 3CPE) of the CPEs 2% limonene (LIM), 1% sodium dodecyl sulfate (SDS) and 0.5% bupivacaine (BUP). Within the exemplary tested formulations, none showed thermoresponsive gelation properties with or without CPEs, except for P188.

P188 had a sol-gel transition temperature >60° C. regardless of the concentration (FIG. 2). Surprisingly, mixing 45% [P188] with 3CPE sharply decreased the gelation temperature of P188 to 26° C. (FIG. 2, FIG. 4), rendering it usefully thermoresponsive in the physiologic temperature range.

45% [P188]-3CPE was a liquid solution with low viscosity (˜0.3 Pa s) at room temperature (FIG. 3B), which could be easily drawn up and extruded through a 20-gauge catheter. 45% [P188]-3CPE gelled ˜13 s after submersion in a 37° C. water bath. 45% [P188]-3CPEs showed much greater gel strength (G′ ˜45 k) at 37° C. compared to 18% P407 (G′ ˜6 k). The thermogelling material with sufficient gel strength had a great advantage in the drug delivery applications, especially in the local drug delivery where a prolonged residence time is required.

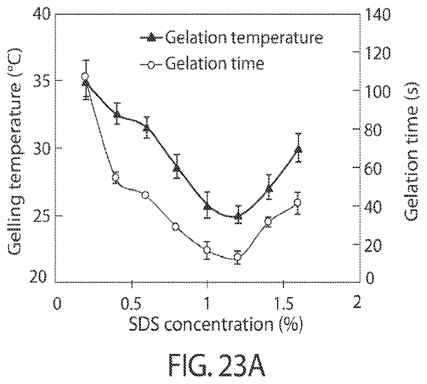

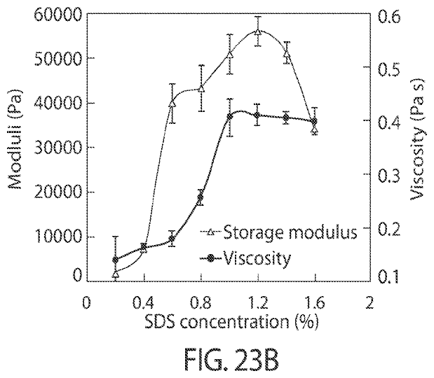

The physical properties of the formulation were studied as a function of the concentrations of the individual components. In the formulation comprising P188-3CPE, increasing the P188 concentration decreased the gelation time and gelation temperature, but increased the viscosity at room temperature and storage modulus (G′) at body temperature (FIGS. 3A-3B). Similarly increasing the concentration of LIM (while keeping the other components of P188-3CPE constant) reduced the gelation time and temperature, and increased room temperature viscosity and gel G′ (FIGS. 12A-12B). In contrast, the addition of 1% SDS completely suppressed the reverse thermal gelation behavior of P188 (FIG. 6A). In the presence of LIM, interestingly, the addition of SDS enhanced the gelation of P188-LIM (i.e., lowered gelation time and temperature, increased G′) at <1.2% SDS), but inhibited gelation at higher concentrations (FIGS. 23A-23B). BUP had a minor effect on the rheological properties of P188 at the concentrations investigated (i.e., 0.2%-4%) (FIG. 6C).

### Trans-Tympanic Permeation Kinetics of the Hydrogel Formulation

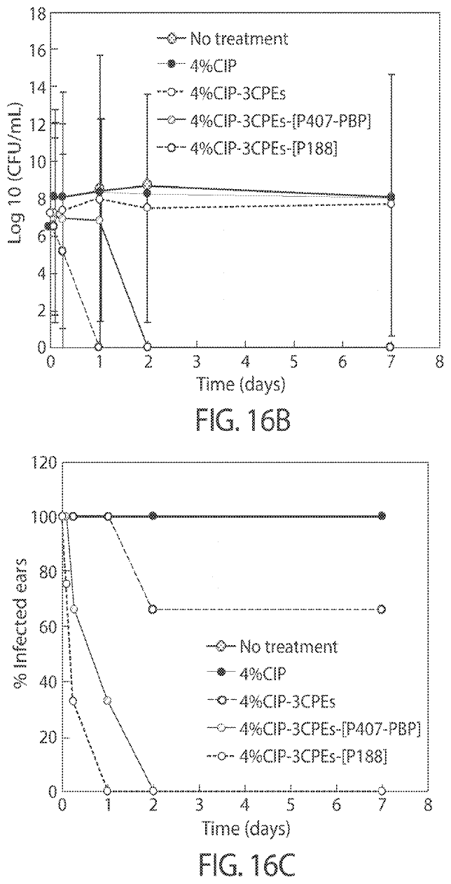

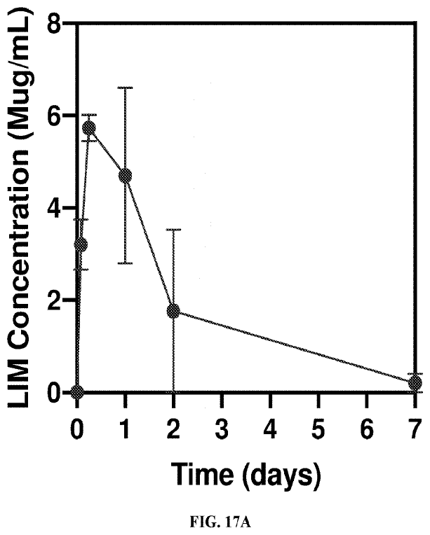

Many of the formulation properties were interdependent: the CPEs are important contributors to trans-tympanic flux of antibiotics, but also allowed gelation of P188 at 37° C. with a strong G′—which could impede flux through the gel. Therefore, the effects of the hydrogel formulation (i.e., P188-3CPEs) on the transport rate of ciprofloxacin (Cip) across the TM were investigated. The rate of drug transport across healthy TMs excised from chinchillas was first evaluated (FIG. 16A). The presence of 3CPE enhanced the flux of Cip over 48 hours from 0.6 mg to 0.82 mg for free drug solution (i.e., 4% Cip-3CPEs) and from 0.43 mg to 0.61 mg for Cip in hydrogel (i.e., 4% Cip-3CPEs-[P188]) after the 48 hours' permeation experiment. Conversely, the inclusion of a hydrogel (i.e., 4% Cip-3CPEs-[P188]) or P188 (i.e., 4% CIP-[P188]) tended to decrease the Cip flux across the TM. This flux retardation effect could be overcome by the addition of 3CPEs, which was indicated from the results that the flux of drug from 4% Cip-3CPEs-[P188] was comparable with the 4% Cip (FIG. 16A). Considering the retention effect of gel on the TM, the drug flux from hydrogel formulation should be still more than adequate for therapeutic effect. An in vivo otitis media (OM) chinchilla model was therefore utilized to mimic the more realistic conditions to demonstrate the presumed principal utility of the hydrogel.

The OM chinchilla was induced by direct inoculation of NTHi into the middle ear of chinchilla, then the infected chinchillas were treated with different test formulations via direct administration through the outer ear canal. A formulation of P407-PBP, a chemically modified P407 that gels with CPEs, was compared with the P188 formulation for OM treatment.

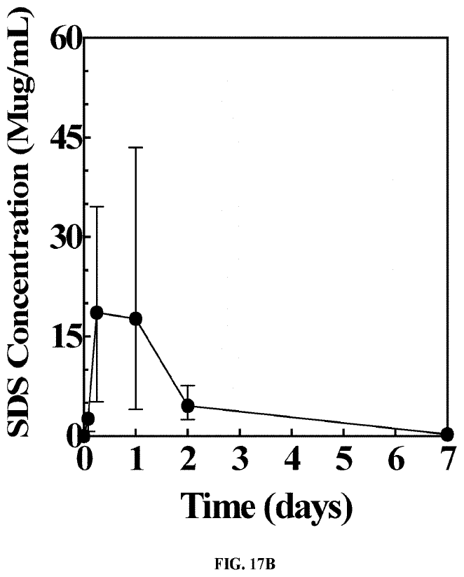

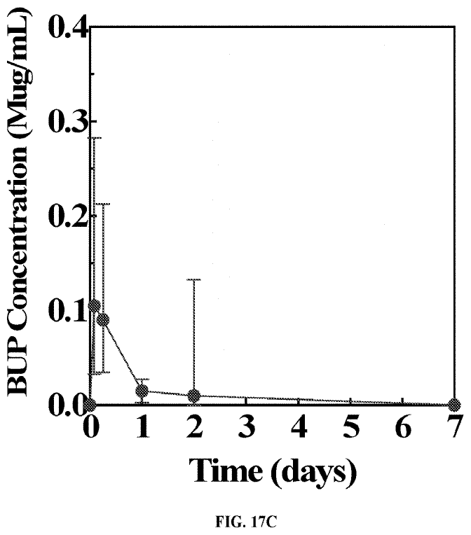

As presented herein, OM was defined as nonzero colony-forming units (CFU) in middle ear fluid (MEF), and a reduction of the bacterial count by 99.9% (i.e., a 3-log reduction) was considered an indication of cure. It was found that the bacterial counts in middle ear fluid from infected animals were not substantively reduced at all time points during the 7-day treatment (FIG. 16B), the infection was detectable in 100% animals and 66.7% animals by day 7 for 4% Cip and 4% Cip-3CPEs treatment, respectively (FIG. 16C). Contrarily, the number of colonies was greatly reduced on day 1 and no colonies were detected on day 2 after the application of the Cip-3CPEs-12%[P407-PBP]. This result is in agreement with the finding from a previous study.¹³ The Cip-3CPEs-[P188] showed even better therapeutic effect, no colonies were detected on day 1 after the application of the formulation (FIGS. 16B-16C). The rapid cure of P188 hydrogel formulations might result from the high concentration of Cip in the middle at the early date points (i.e., 2 h and 6 h). Given the complicated preparation procedures and uncertain safety issue, P188 has advantages compared chemically modified P407 in this application for trans-tympanic drug delivery. In addition, the flux of all CPEs across the TM into the middle ear were detected based on the detected concentration of these CPEs in MEF over time after the P188-3CPEs treatment (FIGS. 17A-17C), which might also account for the good therapeutic effect of P188-3CPEs formulation due to the antimicrobial properties of these CPEs (i.e., SDS and μm).²³⁻²⁴

### Biocompatibility of Hydrogels and Systemic Drug Level

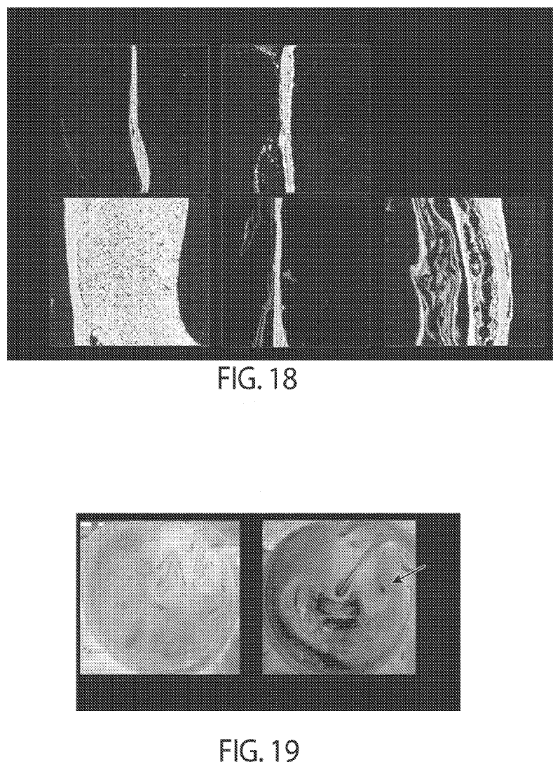

The specific concentration and combination of 3CPEs were chosen since they showed better permeation enhancement on the flux of drug across the TM with no toxic effect in chinchillas and human based on previous reports.²⁵ As presented herein, the biocompatibility of the formulation (P188-3CPEs) was investigated by administration in the ears of healthy/infected chinchillas, followed by histopathology evaluation of the treated TMs after 7 days (a typical treatment duration for OM). After treatment of the formulation (i.e., Cip-3CPEs-[P188]), the H&E-stained sections of both normal/OM infected TMs looked very similar with the TMs from healthy animals, indicating they were biocompatible in the ear, and the infected TM did return to normal status after the gel treatment. Conversely, the TMs were much thicker than healthy TM without gel treatment (i.e., untreated or free Cip treated), which was due to inflammation induced by the alive bacterial (FIG. 18).

One important advantage of the transtympanic formulation presented herein, is the drug can be directly delivered to the target site, which can avoid many side-effects induced by the systemic antibiotic distribution. The blood levels of Cip were investigated by collection of plasma samples at predetermined intervals from the transverse sinuses of chinchillas after hydrogel treatment. These results indicated that no Cip was detectable in the bloodstream at any time point throughout the course of treatment, which provided strong evidence for low or no systemic exposure of antibiotics (Table 1).

### Hydrogel Retention on TM

Improving the local drug retention time is one important strategy for developing the otic formulations (either trans-tympanic or intra-tympanic drug delivery). A recent study found that the recurrence of infection in the middle ear was observed in a long term (i.e., 25 days) OM in vivo study after the application of hydrogel formulation, which was due to the short retention time of the hydrogel on the TM. Additionally, other recent studies suggested that outcomes of OM treatment may be improved when antibiotic treatment regimens lasts 10 or more days.²⁶ Therefore, improving the hydrogel retention on the TM for the prolonged drug delivery is important to achieve an effective therapeutic treatment of ear infection.

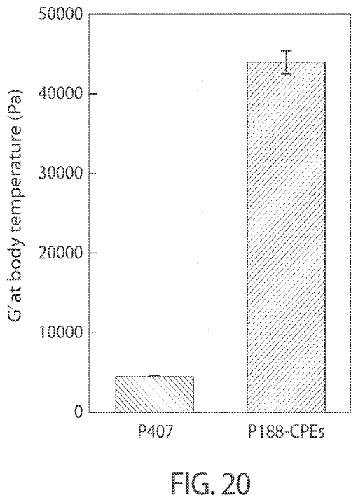

The gel retention properties of formulations with P188-3CPEs were compared with the commonly used thermogelling material (i.e., P407) on the TM. These results showed that a P188-3CPEs formulation could retain on the TM for up to 21 days. In comparison, in this test, the P407 gel was no longer retained on the TM one week after administration (FIG. 19). This was because the hydrogel formed by P188-3CPEs had stronger gel strength (G′) than P407 at 37° C., which may be attributed to more compact packing of the smaller micelles formed from P188-3CPEs than P407 (FIG. 20). It should be noted that the gel strength of P188-3CPEs can be tailored by changing the concentration of each component in the P188-3CPEs formulation to decrease the retention time (e.g., less than 10 days) if the long term retention is not preferred. BUP may also be excluded from this formulation without changing the desired rheological properties of the system.

### Analysis of Results

A local drug platform was developed by combination of CPEs with a poloxamer, P188. The CPEs made P188 gel in the physiological range by changing the micellar structure and micelle formation behavior through an entropy driven process. The P188-3CPEs formulation showed desired viscosity, gelation time and gel strength. This formulation could be easily extruded through a catheter on to the TM at room temperature, which could efficiently deliver drug across the TM and successfully cured the OM in the standard chinchilla animal model through a 7-day course of treatment. The hydrogel formulation was biocompatible in the ear and no systemic exposure of antibiotics was observed after the hydrogel treatment. The gel could retain on the TM for a longer time than P407 due to its stronger mechanical gel strength, which has great advantages in applications when prolonged drug delivery is needed for achieving efficient treatment.

### Experimental

P188 formulation preparation. A certain amount of P188 was dissolved in deionized water (Milli-Q purification system, Millipore) to prepare poloxamer solution. The SDS and bupivacaine were sequentially added in the poloxamer solution in cold room, and stirred at 4° C. for 12 hours. Then the limonene was added into the mixture solution and stirred at 4° C. for at least 2 hours to obtain the final formulation contain 45% P188, 2% limonene, 1% SDS and 0.5% bupivacaine. The concentration in gels is expressed as the mass/volume percentage (w/v), unless specified otherwise. The CPEs concentrations were selected based on those reported to be effective and minimally toxic in previous transdermal studies.²⁷ For the drug loaded formulation, the powdered P188 was dissolved in ciprofloxacin solution and then the CPEs were sequentially added as described above. Hydrogel formulation in scintillation vials was immersed in a water bath kept at 37° C., and the time it took for the hydrogel to firmly stick in the bottle without flow away after overturn the vial was recorded as the gelation time. The effects of different composition on the formation and mechanical properties of the hydrogels were investigated by rheological measurements.

P407-PBP formulation preparation. The poloxamer 407-polybutylphosphoester (P407-PBP) was synthesized based on previously described methods.¹³ Typically, 2-Chloro-2-oxo-1,3,2-dioxaphospholane (COP) (5.0 g, 35 mmol) in anhydrous THF was added to a stirring solution of n-butanol or 2-ethyl-1-butanol (2.6 g, 35 mmol) and trimethylamine (TEA, 3.9 g, 39 mmol) in anhydrous THF at 0° C. dropwise. The reaction mixture was stirred in an ice bath for 12 hours upon completed addition. The reaction mixture was filtered and concentrated filtrate was purified by vacuum distillation under reduced vacuum to get the Butoxy-2-oxo-1,3,2-dioxaphospholane (BP). P407 (8.1 g, 0.56 mmol) and BP (1.0 g, 5.6 mmol) in anhydrous dichloromethane (DCM; 0.5 ml) were mixed in a flame-dried Schlenk flask and flushed with nitrogen gas. A solution of DBU in anhydrous DCM (0.13 g, 0.84 mmol) was added to the stirring solution under nitrogen gas atmosphere. The excess amount of acetic acid in DCM was added to the reaction mixture to quench the reaction. The P407-PBP was purified by precipitation into ether and dried under vacuum to obtain a white powder product. P407-PBP hydrogel formulations were made by dissolving powdered polymers in deionized water and adding CPEs similarly as the P188 formulation.

Rheological Measurements. The gelation temperature and mechanical properties [i.e. the storage (G′) and loss (G″) moduli] of a hydrogel formulation were quantified by linear oscillatory shear rheology measurements using TA instrument Discovery HR-2 (New Castle, Del.). The rheology experiments were run under temperature sweep mode at 5% strain, and the frequency of 10 rad/s. Gelation temperature was taken as the temperature at which the storage modulus (G′) becomes greater than the loss modulus (G″). The shear viscosity was studied by measuring flow curves recorded at shear rates from 0 to 100 s⁻¹.

Differential scanning calorimetry (DSC). DSC was performed on a DSC 6000 (PerkinElmer, USA). Samples of approximately 5-10 mg were weighed into an aluminum container which was hermetically sealed. The rate of heat transfer was measured over the temperature range of 15-70° C. at a scanning rate of 5° C./min.

Isothermal Titration calorimetry (ITC). The microcalorimeter used in the present example was the MicroCal iTC₂₀₀ system (Malvern). The titration was carried out by step-by-step injections of a SDS solution from a 100 μL injection syringe into the sample cell filled with water or polymer solution (˜300 μL). The SDS was either titrated into P188 (PEO-PPO-PEO) or a similar molecular weight PEO molecule (PEG 8000) to understand the binding behavior between SDS and P188. All the measurements were performed at a constant temperature of 25° C.

Ex vivo tympanic membrane (TM) permeation. The Ex vivo TM permeation rate of antibiotics was determined with auditory bullae harvested from healthy chinchillas. All formulations (200 μL) were applied into the bullae kept at 37° C. and deposited onto the TMs. The bullae were placed with the ear canal side facing up in a 12-well plate with 3 mL phosphate buffered saline (PBS) in each well. Permeation of ciprofloxacin across TM into the receiving chamber after 0.5, 1, 2, 6, 12, 24 and 48 hours at 37° C. was quantified using liquid chromatography mass spectrometry (LC-MS). Detailed information regarding TM harvesting, electrical resistance measurement and configuration of the ex vivo permeation experiment can be found in a previous study.²⁸

NTHi OM chinchilla model and pharmacokinetics. All procedures and manipulations were performed using sedation analgesia under isoflurane chamber induction followed by mask inhalation of 1-3% isoflurane in accordance with approved IACUC protocols at Boston Children's Hospital. Baseline plasma samples were obtained through the cephalic sinus 24 hours before bacterial inoculation. Isolates of NTHi grown to the mid-log phase were diluted in Hanks' balanced salt solution (HBSS), and about 25 to 75 CFU in 100 ml were directly introduced into each middle ear through the dorsal aspect of the tympanic bullae under aseptic conditions. Once the middle ear infection was confirmed using methods described previously, the chinchillas were administrated with 200 μL test formulations via a soft catheter (20-gauge, 1.8-inch) on to the TM through their external ear canal.¹³ Then the middle ear fluids (MEF) were obtained with a 22-gauge angiocatheter connected syringe after 2, 6, 24, 48, and 168 hours based on the previously described methods.¹³ The serial 10-fold dilutions of MEF were prepared in HBSS and then one hundred microliters of each dilution was plated onto the blood agar for bacterial counting. Serial plasma samples were also obtained through the cephalic sinus during the experiment to determine systemic drug levels.

Histopathology. Hydrogel formulations were administered onto the tympanic membrane of live healthy/OM chinchillas. After 7 days later, they were euthanized as described elsewhere. The TMs were then excised and immediately fixed in 10% neutral buffered formalin overnight. The sectioned TMs (˜10 μm thick) were stained with hematoxylin and eosin, and evaluated by light microscopy in a blinded fashion.

Statistical analysis. The results are reported as averages and standard deviations, and the differences among treatments were calculated based on an analysis of variance (ANOVA) and a post-hoc Duncan test with a confidence level of 95%. These analyses were carried out using statistical analysis software (SPSS, IBM Corporation, Armonk, N.Y., USA).

### REFERENCES IN EXAMPLE 5

1. Michael T. Cook, and Marc B. Brown. “Polymeric gels for intravaginal drug delivery.” Journal of Controlled Release 270 (2018): 145-157.
2. Gilles Dumortier, Jean Louis Grossiord, Florence Agnely, and Jean Claude Chaumeil. “A review of poloxamer 407 pharmaceutical and pharmacological characteristics,” Pharmaceutical research 23, no. 12 (2006): 2709-2728.
3. Guoguang Niu, Fengyi Du, Li Song, Hongbin Zhang, Jun Yang, Hui Cao, Yudong Zheng et al. “Synthesis and characterization of reactive poloxamer 407s for biomedical applications.” Journal of controlled release 138, no. 1 (2009): 49-56.
4. Lin Zhang, Daniel L. Parsons, Christine Navarre, and Uday B. Kompella. “Development and in-vitro evaluation of sustained release poloxamer 407 (P407) gel formulations of ceftiofur.” Journal of controlled release 85, no. 1-3 (2002): 73-81.
5. Karsten Milder, Eric Lehner, Arne Liebau, and Stefan K. Plontke. “Controlled drug release to the inner ear: Concepts, materials, mechanisms, and performance.” Hearing research 368 (2018): 49-66.
6. Mohamad A. Abou-Shamat, Jesus Calvo-Castro, Jacqueline L. Stair, and Michael T. Cook. “Modifying the properties of thermogelling poloxamer 407 solutions through covalent modification and the use of polymer additives.” Macromolecular Chemistry and Physics 220, no. 16 (2019): 1900173.
7. Hyun Jung Chung, Yuhan Lee, and Tae Gwan Park. “Thermo-sensitive and biodegradable hydrogels based on stereocomplexed Pluronic multi-block copolymers for controlled protein delivery.” Journal of controlled release 127, no. 1v (2008): 22-30.
8. Kyung Min Park, Sang Young Lee, Yoon Ki Joung, Jae Sik Na, Myung Chul Lee, and Ki Dong Park. “Thermosensitive chitosan-Pluronic hydrogel as an injectable cell delivery carrier for cartilage regeneration.” Acta biomaterialia 5, no. 6 (2009): 1956-1965.
9. Yuhan Lee, Hyun Jung Chung, Sangho Yeo, Cheol-Hee Ahn, Haeshin Lee, Phillip B. Messersmith, and Tae Gwan Park. “Thermo-sensitive, injectable, and tissue adhesive sol-gel transition hyaluronic acid/pluronic composite hydrogels prepared from bin-inspired catechol-thiol reaction.” Soft Matter6, no. 5 (2010): 977-983.
10. Daniel Cohn, Alejandro Sosnik, and Avraham Levy. “Improved reverse thermoresponsive polymeric systems.” Biomaterials 24, no. 21 (2003): 3707-3714.
11. Seung Yong Lee, Yuhan Lee, Su Young Chae, Tae Gwan Park, and Cheol-Hee Ahn. “Blends of oppositely charged PEG-PPG-PEG copolymers displaying improved physical thermogelling properties.” Macromolecular Chemistry and Physics 211, no. 6 (2010): 692-697.
12. Vasudevanpiliai Biju. “Chemical modifications and bioconjugate reactions of nanomaterials for sensing, imaging, drug delivery and therapy.” Chemical Society Reviews 43, no. 3 (2014): 744-764.
13. Rong Yang, Vishakha Sahharwal, Obiajulu S. Okonkwo, Nadya Shlykova, Rong Tong, Lily Yun Lin, Weiping Wang et al. “Treatment of otitis media by transtympanic delivery of antibiotics.” Science translational medicine 8 no. 356 (2016): 356ra120-356ra120.
18. Rouja Ivanova, Björn Lindman, and Paschalis Alexandridis. “Effect of pharmaceutically acceptable glycols on the stability of the liquid crystalline gels formed by poloxamer 407 in water.” Journal of colloid and interface science 252, no. 1 (2002): 226-235.
21. María Pérez-Vázquez, Federico Roman, Belen Aracil, Rafael Cantön, and José Campos. “In vitro activities of garenoxacin (BMS-284756) against Haemophilus influenzae isolates with different fluoroquinolone susceptibilities.” Antimicrobial agents and chemotherapy 47, no. 11 (2003): 3539-3541.
22. Yoichi Hirakata, Kaori Ohmori, Miwako Mikuriya, Takeshi Saika, Kaoru Matsuzaki, Miyuki Hasegawa, Masumitsu Hatta et al. “Antimicrobial activities of piperacillin-tazobactam against Haemophilus influenzae isolates, including β-lactamase-negative ampicillin-resistant and μ-lactamase-positive amoxicillin-clavulanate-resistant isolates, and mutations in their quinolone resistance-determining regions.” Antimicrobial agents and chemotherapy 53, no. 10 (2009): 4225-4230.
23. S F van Vuuren, and Alvaro M. Viljoen, “Antimicrobial activity of limonene enantiomers and 1,8-cineole alone and in combination.” Flavour and fragrance journal 22, no. 6 (2007): 540-544.
24. Sonar Chouhan, Kanika Sharma, and Sanjay Guleria. “Antimicrobial activity of some essential oils present status and future perspectives.” Medicines 4, no. 3 (2017): 58.
25. Emmanuel John Simons. “Chemical penetration enhancers and in situ-forming reservoirs for trans-tympanic drug delivery: progress toward improved treatment of Otitis media.” PhD diss., Massachusetts Institute of Technology, 2008.
26. Alejandro Hoberman, Jack L. Paradise, Howard E. Rockette, Diana H. Kearney, Sonika Bhatnagar, Timothy R. Shope, Judith M. Martin et al. “Shortened antimicrobial treatment for acute otitis media in young children,” New England Journal of Medicine 375, no. 25 (2016): 2446-2456.
27. Pankaj Karande, Amit Jain, Kaitlin Ergun, Vincent Kispersky, and Samir Mitragotri. “Design principles of chemical penetration enhancers for transdermal drug delivery.” Proceedings of the National Academy of Sciences 102, no. 13 (2005): 4688-4693.
28. Xiaojuan Khoo, Emmanuel J. Simons, Homer H. Chiang, Julia M. Hickey, Vishakha Sabharwal, Stephen I. Pelton, John J. Rosowski, Robert Langer, and Daniel S. Kahane. “Formulations for trans-tympanic antibiotic delivery.” Biomaterials 34, no. 4 (2013): 1281-1288.

### Example 6

To prepare the ceftriaxone (CTX) formulation, the P188-CPEs were first formulated (without CTX) to maximize trans-tympanic flux of antibacterial activity (TTFAA) by rationally increasing the concentration of CPEs (i.e., SDS and LIM). Four strong formulations (formulations Nos. 1-4) were identified that contain a relatively high amount of CPEs while maintaining the desired rheological properties, such as short gelation time, low viscosity and gelation in a physiological temperature range. The CPEs' concentration range was then fixed based on these formulations, and then the TTFAA was further maximized by maximizing the amount of CTX in the formulation. The exemplary formulations described in Example 6 are shown in Table 4 below. Formulations 1-9 from Table 4 were prepared as described above and herein.

### Rheological Properties of Formulations in Table 4

The formulations of Table 4 were analyzed to obtain the depicted data on rheological properties in Table 4. The experiments and measurements for obtaining the rheological properties shown in Table 4 (gelation (gel) temperature, mechanical properties, gel time, viscosity at 25° C., G′ at 37° C.) were conducted as disclosed above in Example 2 and in the section above titled “Rheological Measurements” (via linear oscillatory shear rheology measurements).

### Ex Vivo Study of Tympanic Membrane Permeation in Chinchilla Animal Model for Formulations in Table 4

An ex vivo study in a chinchilla animal model was conducted using the formulations in Table 4. The experiments for obtaining the ex vivo flux data shown in Table 4 below were conducted as disclosed in the section above titled “Ex vivo tympanic membrane (TM) permeation” (for a chinchilla animal model).

### In Vivo Study of Flux and Cure Rate in Chinchilla Animal Model for Formulations in Table 4

An in vivo study in a chinchilla animal model was conducted using the formulations in Table 4. The experiments for obtaining the in vivo data (in vivo flux, cure rate) shown in Table 4 below were conducted as follows. Isolated NTHi were introduced to chinchillas through the nasopharynx. After 48 hours, NTHi was then directly introduced into each middle ear of chinchillas through the dorsal aspect of the tympanic bullae under aseptic conditions. Once the middle ear infection was confirmed using methods described previously, the chinchillas were administrated with 200 μL test formulations via a soft catheter (20-gauge, 1.8-inch) on to the TM through their external ear canal. Next, the middle ear fluids (MEF) were obtained with a 22-gauge angiocatheter connected syringe after 3-4 hours, 24, 48, and 168, 240 and 504 hours. The serial 10-fold dilutions of MEF were prepared in HBSS and then one hundred microliters of each dilution was plated onto the blood agar for bacterial counting. Serial plasma samples were also obtained through the cephalic sinus during the experiment to determine the systemic drug levels.

To obtain the data on tympanic membrane dwell time in the chinchilla animal model (shown in Table 4 below), the following experimental procedure was conducted. On day 0 of the experiment, the animals were first anesthetized. Once the anesthesia was confirmed, 0.2 mL of formulation will be delivered to each ear, sequentially via syringe and 20 G soft-tipped Insyte autoguard catheter. In order to assess the length of time the formulation stays on the tympanic membrane, on specific days after drug was applied—either 1, 2, 3, 4, 7, 10, or 21 days after application—groups of animals were anesthetized. The presence/absence of formulation on the tympanic membrane was observed using the otoscopy through the external ear canal.

TABLE 4
The composition and properties of the ceftriaxone-hydrogel formulations.

| | | | | | | | | | Performance in animal studies | | | |
|---|---|---|---|---|---|---|---|---|---|---|---|---|
| | Concentration of components | | | | Rheological properties | | | | TM | Ex | In | |
| | P188 | SDS | LIM | CTX | Gel | Gel | Viscosity | G′ at | dwell | vivo | vivo | Cure |
| | (%, | (%, | (%, | (%, | temp | time | at 25° C. | 37° C. | time | flux | flux | rate |
| No. | w/v) | w/v) | w/v) | w/v) | (° C.) | (s) | (Pa s) | (kPa) | (d) | (mg) | (ug/mL) | (%) |
| 1 | 35 | 2.4 | 5 | — | 27 | 8 | 0.311 | 44.9 | — | — | — | — |
| 2 | 35 | 2.6 | 5 | — | 30 | 13 | 0.289 | 27.8 | — | — | — | — |
| 3 | 35 | 2.8 | 4 | — | 35.5 | 31 | 0.35 | 7.7 | — | — | — | — |
| 4 | 35 | 3 | 4 | — | 34 | 28 | 0.336 | 8.3 | — | — | — | — |
| 5 | 23.5 | 2.6 | 5 | 42.6 | 34 | 31 | 0.598 | 5.3 | — | 3.5 | — | — |
| 6 | 21.375 | 2.16 | 4.05 | 28 | 34.5 | 19 | 0.21 | 7.9 | 3 | 2.17 | 3806 | 75% |
| 7 | 25 | 2.16 | 4.05 | 4 | 31.5 | 18 | 0.357 | 8.4 | — | 0.77 | 851 | 50% |
| 8 | 23 | 2.4 | 5 | 21 | 27 | 11 | 0.43 | 17 | 1-6 | — | — | — |
| 9 | 24 | 2.6 | 5 | 20 | 31 | 12 | 0.37 | 10 | 21 | — | 2063 | 100% |
| 10 | 26 | 2.6 | 5 | 17 | 30 | 10 | 0.46 | 20 | 3-6 | — | — | — |
| 11 | 35 | 2 | 2 | 27 | 33 | 13 | 0.453 | 17 | 10-15 | 6.05 | — | 100% |

Note:
G′ is the storage modulus of the formulation, TM dwell time is the retention time of gel on the tympanic membrane (TM) after administration, Ex vivo flux is the amount of drug flux across the TM after 48 hours, In vivo flux is the peak concentration of drug in the middle ear fluid after formulation administration, cure rate is the otitis media clearance rate in the NTHi infected chinchilla. SDS: sodium dodecyl sulfate, LIM: limonene, CTX: ceftriaxone sodium.

The following summarizes the data from these experiments (see Table 4, showing summary of exemplary formulations):
1. 42.6% CTX (formulation No. 5) was the highest amount of this drug that can be dissolved in the P188-CPEs formulation while maintaining the desired rheological properties.
2. 28% CTX (formulation No. 6) is the highest amount of this drug that can be dissolved in the P188-CPEs formulation as a powder form within a short time (<1 minute) while maintaining the desired rheological properties.
3. The Ex vivo studies (on a chinchilla animal model, as described above) showed that the amount of cumulative CTX increased as increasing amount of drug initially loaded in the formulation (i.e., 42.6% CTX>28% CTX>4% CTX).
4. In the in vivo study (on a chinchilla animal model, as described above), both 28% CTX (formulation No. 6) and 4% CTX (formulation No. 7) formulations were found to clear the otitis media (OM) in the NTHi infected chinchillas after 4 hours' treatment. However, the recurrence of infection was observed after 22 days for the animals treated with 28% CTX formulation and 10 days for 4% CTX formulation. The OM cure rate is 75% and 50%, respectively.
5. In the in vivo study (on a chinchilla animal model, as described above), the drug level in the middle ear peaked after 4 hours in the animals treated with the formulations with either 28% CTX (formulation No. 6) or 4% CTX (formulation No. 7) (3800 ug/mL for 28% CTX and ˜850 ug/mL for 4% CTX formulation).
6. The recurrence of infection from formulation No. 6 and No. 7 might be attributed to the low retention time of both formulation (˜1 day) on the TM because of relatively low gel strength (G′<6 kpa) of both formulations at 37° C.
7. The formulations were further optimized to increase the gel strength without compromising other advantageous rheological properties (i.e., short gelation time and low viscosity) and TTFAA. Three formulations were identified including: 21% CTX-23% P188-5% LIM-2.4% SDS (formulation No. 8, G′ ˜17 kpa), 20% CTX-24% P188-5% LIM-2.6% SDS (formulation No. 9, G′ ˜14 kpa) and 17% CTX-26% P188-5% LIM-2.6% SDS (formulation No. 10, G′ ˜20 kpa).
8. The 20% CTX (formulation No. 9) formulation showed good performance (retaining at least two weeks) in a 21-day hydrogel retention study on the TM of healthy chinchillas.
9. The 20% CTX formulation (formulation No. 9) could successfully clear the OM in the NTHi infected chinchillas over a 21-day course of treatment without recurrence of infection. The drug level in middle ear peaked after 4 hours (2063 ug/mL).
10. The P188-CPEs-CTX formulation (formulation No. 11) was also prepared with a low concentration of CPEs (i.e., 2% SDS and 2% LIM). The Ex vivo studies showed that the amount of cumulative CTX was around 6.05 mg after 48 hours, which was the highest drug flux amount obtained from these Ex vivo experiments. This formulation could also successfully clear the OM in the NTHi infected chinchillas over a 21-day course of treatment without recurrence of infection.

### REFERENCES

1. Yang, R.; Okonkwo, O. S.; Zurakowski, D.; Kohane, D. S., Synergy between chemical permeation enhancers and drug permeation across the tympanic membrane. Journal of Controlled Release 2018, 289, 94-101.
2. Khoo, X.; Simons, E. J.; Chiang, H. H.; Hickey, J. M.; Sabharwal, V.; Pelton, S. I.; Rosowski, J. J.; Langer, R.; Kohane, D. S., Formulations for trans-tympanic antibiotic delivery. Biomaterials 2013, 34 (4), 1281-1288.
3. Pérez-Vázquez, M.; Roman, F.; Aracil, B.; Cantón, R.; Campos, J., In vitro activities of garenoxacin (BMS-284756) against Haemophilus influenzae isolates with different fluoroquinolone susceptibilities. Antimicrobial agents and chemotherapy 2003, 47 (11), 3539-3541.
4. Yang, R.; Sabharwal, V.; Okonkwo, O. S.; Shlykova, N.; Tong, R.; Lin, L. Y.; Wang, W.; Guo, S.; Rosowski, J. J.; Pelton, S. I., Treatment of otitis media by transtympanic delivery of antibiotics. Science translational medicine 2016, 8 (356), 356ra120-356ra120.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A composition comprising:
(a) a therapeutic agent or a combination of therapeutic agents;
(b) a permeation enhancer or a combination of permeation enhancers, wherein the permeation enhancer or combination of permeation enhancers increases the flux of the therapeutic agent or combination of therapeutic agents across a barrier; and
(c) a block copolymer comprising poloxamer P188;
wherein:
the permeation enhancer or combination of permeation enhancers comprises sodium dodecyl sulfate, limonene, and/or bupivacaine, and
the sodium dodecyl sulfate comprises between about 0.2% and 3.2% of the composition by weight per volume composition;
when present, the bupivacaine comprises between about 0.2% and 2.0% of the composition by weight per volume composition;
the limonene comprises between about 0.5% and 7.0% of the composition by weight per volume composition; and
the poloxamer P188 comprises between about 18% and about 62% of the composition by weight per volume composition;
wherein:
the composition forms a gel at temperatures above a phase transition temperature; and
the phase transition temperature is less than about 37° C.

2. The composition of claim 1, wherein:
(c) the block copolymer is poloxamer P188;
the permeation enhancer or combination of permeation enhancers comprises sodium dodecyl sulfate, limonene, and/or bupivacaine, and
the sodium dodecyl sulfate comprises between about 0.5% and 3.2% of the composition by weight per volume composition;
the bupivacaine comprises between about 0.25% and 0.75% of the composition by weight per volume composition;
the limonene comprises between about 1.5% and 6.0% of the composition by weight per volume composition; and
the poloxamer P188 comprises between about 20% and about 50% of the composition by weight per volume composition;
wherein:
the composition forms a gel at temperatures above a phase transition temperature; and
the phase transition temperature is between about 20° C. and about 37° C.

3. The composition of claim 1 or 2, wherein the composition comprises the permeation enhancer limonene.

4. The composition of any one of claims 1-3, wherein the composition comprises the permeation enhancers sodium dodecyl sulfate and limonene.

5. The composition of any one of claims 1, 3, or 4, wherein the permeation enhancers consist of sodium dodecyl sulfate and limonene.

6. The composition of any one of claims 1-4, wherein the composition comprises the permeation enhancers sodium dodecyl sulfate, limonene, and bupivacaine.

7. The composition of any one of claims 1-6, wherein the composition comprises between about 1.5-3.2% wt/vol of sodium dodecyl sulfate.

8. The composition of any one of claims 1-7, wherein the composition comprises between about 0.2-1.0% wt/vol of sodium dodecyl sulfate.

9. The composition of any one of claims 1-7, wherein the composition comprises between about 1.0-2.7% wt/vol of sodium dodecyl sulfate.

10. The composition of any one of claims 1-7 or 9, wherein the composition comprises between about 1.8-2.7% wt/vol of sodium dodecyl sulfate.

11. The composition of any one of claims 1-7, 9, or 10, wherein the composition comprises between about 2.0-2.6% wt/vol of sodium dodecyl sulfate.

12. The composition of any one of claims 1-7, wherein the composition comprises between about 0.5-1.5% wt/vol of sodium dodecyl sulfate.

13. The composition of any one of claims 1-9 or 12, wherein the composition comprises about 1.0% wt/vol of sodium dodecyl sulfate.

14. The composition of any one of claims 1, 3, 4, or 6-13, wherein the composition comprises between about 0.5-1.25% wt/vol of bupivacaine.

15. The composition of any one of claims 1-4 or 6-13, wherein the composition comprises between about 0.3-0.6% wt/vol of bupivacaine.

16. The composition of any one of claims 1, 3, 4, or 6-13, wherein the composition comprises about 1.0-1.25% wt/vol of bupivacaine.

17. The composition of any one of claims 1-4 or 6-15, wherein the composition comprises about 0.5% wt/vol of bupivacaine.

18. The composition of claim 1, 3, 4, 6-14, or 16, wherein the composition comprises about 1.0% wt/vol of bupivacaine.

19. The composition of any one of claims 1-18, wherein the composition comprises between about 4.0-6.0% wt/vol of limonene.

20. The composition of any one of claims 1-19, wherein the composition comprises between about 4.0-5.0% wt/vol of limonene.

21. The composition of any one of claims 1-18, wherein the composition comprises about 0.5-3.5% wt/vol of limonene.

22. The composition of any one of claims 1-17 or 21, wherein the composition comprises about 2.0% wt/vol of limonene.

23. The composition of any one of claims 1-20, wherein the composition comprises about 4.0% wt/vol of limonene.

24. The composition of any one of claims 1-20, wherein the composition comprises about 5.0% wt/vol of limonene.

25. The composition of any one of claims 1-24, wherein the composition comprises between about 21.0-37.0% wt/vol of poloxamer P188.

26. The composition of any one of claims 1-24, wherein the composition comprises between about 30.0-50.0% wt/vol of poloxamer P188.

27. The composition of any one of claims 1-26, wherein the composition comprises between about 34.0-37.0% wt/vol of poloxamer P188.

28. The composition of any one of claims 1-24 or 26, wherein the composition comprises between about 40.0-50.0% wt/vol of poloxamer P188.

29. The composition of any one of claims 1-27, wherein the composition comprises about 21.0% wt/vol, about 23.0% wt/vol, about 24.0% wt/vol, about 25.0% wt/vol, about 26.0% wt/vol, or about 35.0% wt/vol of poloxamer P188.

30. The composition of any one of claims 1-26 or 28, wherein the composition comprises between about 45.0% wt/vol of poloxamer P188.

31. The composition of any one of claims 1-30, wherein the phase transition temperature is between about 24.0° C. and about 37.0° C.

32. The composition of any one of claims 1-31, wherein the phase transition temperature is between about 29.0° C. and about 36.5° C.

33. The composition of any one of claims 1-30, wherein the phase transition temperature is between about 20.0° C. and about 30.0° C.

34. The composition of any one of claims 1-30 or 33, wherein the phase transition temperature is about 25.0° C.

35. The composition of any one of claims 1-34, wherein the phase transition temperature is about 25.0° C., about 27.0° C., about 30.0° C., about 31.0° C., about 33.0° C., about 34.0° C., or about 35.5° C.

36. The composition of any one of claims 1-35, wherein the therapeutic agent is an antibiotic agent, anesthetic agent, anti-inflammatory agent, analgesic agent, anti-fibrotic agent, anti-sclerotic agent, anticoagulant agent, or diagnostic agent.

37. The composition of claim 36, wherein the antibiotic agent is a fluoroquinolone or a beta lactam antibiotic.

38. The composition of claim 36 or 37, wherein the antibiotic agent is selected from the group consisting of ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole.

39. The composition of any one of claims 36-38, wherein the antibiotic agent is ciprofloxacin or ceftriaxone.

40. The composition of any one of claims 36-39, wherein the composition comprises between about 3.0-50.0% wt/vol of the antibiotic agent that is ciprofloxacin or ceftriaxone.

41. The composition of any one of claims 36-39, wherein the antibiotic agent is ciprofloxacin.

42. The composition of claim 41, wherein the composition comprises between about 1.0-5.0% wt/vol of ciprofloxacin.

43. The composition of any one of claims 36-38, wherein the antibiotic is ciprofloxacin, gatifloxacin, ceftriaxone, gemifloxacin, moxalactam, levofloxacin, meropenem, or ampicillin.

44. The composition of any one of claims 1-43, wherein the composition comprises:
about 1.5-3.2% wt/vol of sodium dodecyl sulfate;
about 2.0-5.0% wt/vol of limonene;
about 21.0-37.0% wt/vol of poloxamer P188; and
about 4.0-45.0% wt/vol of antibiotic.

45. The composition of any one of claims 1-44, wherein the composition comprises:
about 2.0-3.1% wt/vol of sodium dodecyl sulfate;
about 4.0-5.0% wt/vol of limonene;
about 21.0-36.0% wt/vol of poloxamer P188; and
about 16.0-43.0% wt/vol of antibiotic.

46. The composition of any one of claims 1-44, wherein the composition comprises:
about 2.0-3.1% wt/vol of sodium dodecyl sulfate;
about 4.0-5.0% wt/vol of limonene;
about 21.0-36.0% wt/vol of poloxamer P188; and
about 16.0-43.0% wt/vol of ceftriaxone.

47. The composition of any one of claims 1-43, wherein the composition comprises:
about 1.5-3.2% wt/vol of sodium dodecyl sulfate;
about 3.0-5.0% wt/vol of limonene;
about 34.0-37.0% wt/vol of poloxamer P188; and
about 2.0-5.0% wt/vol of antibiotic.

48. The composition of any one of claims 1-43 or 47, wherein the composition comprises:
about 1.0% wt/vol of sodium dodecyl sulfate;
about 0.5% wt/vol of bupivacaine;
about 2.0% wt/vol of limonene; and
about 45.0% wt/vol of poloxamer P188.

49. The composition of any one of claims 1-43, 47, or 48, wherein the composition comprises:
about 1.0% wt/vol of sodium dodecyl sulfate;
about 0.5% wt/vol of bupivacaine;
about 2.0% wt/vol of limonene;
about 45.0% wt/vol of poloxamer P188; and
about 2.0-5.0% wt/vol of ciprofloxacin.

50. A pharmaceutical composition comprising a composition of any one of claims 1-49, and optionally a pharmaceutically acceptable excipient.

51. The pharmaceutical composition of claim 50, wherein the pharmaceutical composition comprises a therapeutically effective amount of the composition for use in treating a disease or condition in a subject in need thereof.

52. A method of treating a disease or condition in a subject in need thereof, the method comprising administering to the subject a therapeutically effective amount of a composition of any one of claims 1-49, or pharmaceutical composition of claim 50 or 51.

53. The pharmaceutical composition of claim 51, wherein the disease is an infectious disease.

54. The pharmaceutical composition of claim 53, wherein the infectious disease is a bacterial infection.

55. The pharmaceutical composition of claim 54, wherein the bacterial infection is an H. influenzae, S. pneumoniae, or M. catarrhalis infection.

56. The pharmaceutical composition of claim 51, wherein the disease is an ear disease.

57. The pharmaceutical composition of claim 53, wherein the infectious disease is otitis media.

58. The method of claim 52, wherein the disease is an infectious disease.

59. The method of claim 58, wherein the infectious disease is a bacterial infection.

60. The method of claim 59, wherein the bacterial infection is an H. influenzae, S. pneumoniae, or M. catarrhalis infection.

61. The method of claim 52, wherein the disease is an ear disease.

62. The method of claim 58, wherein the infectious disease is otitis media.

63. A method of delivering a composition of any one of claims 1-49, the method comprising administering the composition to an ear canal of a subject.

64. The method of claim 63, wherein the composition contacts the surface of a tympanic membrane.

65. The method of claim 64, wherein the administering comprises placing drops of the composition into the ear canal, or placing a dose of the composition into the ear canal using a catheter.

66. The method of any one of claims 63-65, wherein the administering comprises using an applicator to place the composition into the ear canal.

67. The method of any one of claims 63-66, wherein the administering comprises placing the composition into the ear canal with a syringe.

68. Use of a composition to treat and/or prevent a disease or condition in a subject in need thereof, the use comprising administering to the subject a therapeutically effective amount of a composition of any one of claims 1-49, or pharmaceutical composition of claim 50 or 51.

69. A kit for treating an ear disease and/or condition associated with an ear disease comprising a container, a composition of any one of claims 1-49, and instructions for administering the composition to a subject in need thereof.

70. The kit of claim 69, further comprising a dropper, syringe, or catheter.

71. The kit of claim 69 or 70, comprising formulating an antibiotic from a powder form in the composition of any one of claims 1-49.
